(19) United States
(12) Patent Application Publication
(10) Pub. No.: US 2021/0128690 A1
(43) Pub. Date: May 6, 2021

(54) METHODS OF ADMINISTERING HEPCIDIN

(71) Applicant: La Jolla Pharmaceutical Company, San Diego, CA (US)

(72) Inventors: Lakhmir Chawla, San Diego, CA (US); George Tidmarsh, Portola Valley, CA (US)

(21) Appl. No.: 16/471,417

(22) PCT Filed: Dec. 19, 2017
(86) PCT No.: PCT/US2017/067400

§ 371 Date: Jun. 19, 2019

Related U.S. Application Data
(60) Provisional application No. 62/527,354, filed on Jun. 30, 2017.
(60) Provisional application No. 62/474,347, filed on Mar. 21, 2017.
(60) Provisional application No. 62/443,088, filed on Jan. 6, 2017.
(60) Provisional application No. 62/436,070, filed on Dec. 19, 2016.

(51) Int. Cl. A61K 38/22; A61K 45/06

(52) U.S. Cl. CPC A61K 38/22; A61K 45/06

(57) ABSTRACT

The present disclosure relates to the use of hepcidin in therapeutic methods for the treatment of various conditions in which decreasing serum iron concentration may be beneficial.

### RELATED APPLICATIONS

This application claims the benefit of priority to U.S. Provisional Patent Application Ser. No. 62/436,070, filed on Dec. 19, 2016, U.S. Provisional Patent Application Ser. No. 62/443,088, filed on Jan. 6, 2017, U.S. Provisional Patent Application Ser. No. 62/474,347, filed on Mar. 21, 2017, and U.S. Provisional Patent Application Ser. No. 62/527,354, filed on Jun. 30, 2017, each of which are herein incorporated by reference in their entireties.

### BACKGROUND

Iron is an essential element required for growth and survival of almost every organism. In mammals, the iron balance is primarily regulated at the level of duodenal absorption of dietary iron. Following absorption, ferric iron is loaded into apo-transferrin in the circulation and transported to the tissues, including erythroid precursors, where it is taken up by transferrin receptor-mediated endocytosis. Reticuloendothelial macrophages play a major role in the recycling of iron from the degradation of hemoglobin of senescent erythrocytes, while hepatocytes contain most of the iron stores of the organism in ferritin polymers.

In the case of iron deficiency, the pathophysiological consequences of gene defects identified are well understood because they usually result in loss of function of proteins directly involved in the pathway of iron absorption. The proteins include the iron transporters DMT1 (also called Nramp2 or DCT1), ferroportin (also called IREG1 or MTP1), and copper oxidases coupled to ferroportin, namely ceruloplasmin and haephastin. Additionally, several abnormalities associated with genetic iron overload have led to the identification of other proteins, but the functional role of these proteins remains poorly understood. In humans, hereditary hemochromatosis (HH) is a common autosomal recessive genetic disease caused by hyperabsorption of dietary iron leading to an iron overload in plasma and organs, including the pancreas, liver, and skin, resulting in damage caused by iron deposit.

Hemochromatosis is usually caused by a mutation in the HLA-linked hemochromatosis gene (named HFE) located on chromosome 6p, and most symptomatic patients are homozygous for the C282Y mutation. Additionally, other loci have been implicated in hereditary hemochromatosis: a nonsense mutation in the transferrin receptor 2 gene (TFR2) on 7q has been reported in two HH non-HLA-linked families, and a locus for juvenile hemochromatosis has recently been mapped to chromosomal arm lq (HFE2). Finally, although it has long been known that iron absorption is regulated in response to the level of body iron stores and to the amount of iron needed for erythropoiesis, the molecular nature of the signals that program the intestinal cells to adjust iron absorption remains unknown.

### SUMMARY

The present disclosure relates to the use of hepcidin or mini-hepcidin in therapeutic methods for the treatment of various conditions in which decreasing serum iron concentration may be beneficial. In some aspects, the invention relates to a method for treating a condition in a subject, comprising administering a composition comprising hepcidin or mini-hepcidin to the subject. In some aspects, the invention relates to a method for decreasing the absorption of dietary iron in a subject, comprising administering a composition comprising hepcidin or mini-hepcidin to the subject. In some aspects, the invention relates to a method for reducing the serum iron concentration of a subject, comprising administering a composition comprising hepcidin or mini-hepcidin to the subject.

In some aspects, provided herein are methods of treating or preventing disorders of iron homeostasis, e.g., hemochromatosis, α-thalassemia, thalassemia intermedia, β-thalassemia, sickle cell disease, refractory anemia, sideroblastic anemia, Diamond-Blackfan anemia (DBA), or hemolytic anemia. Existing interventions for these conditions are often burdensome or time-consuming for the patient, and fail in many cases to provide durable improvements in patient health and well-being.

Hepcidin alters the body's response to infectious disease, conferring benefits beyond any direct effect that hepcidin may have on the infectious agent. Such benefits include moderating increases in blood iron levels caused by the death of red blood cells, sequestering blood iron and thus limiting access of the infectious agent to this nutrient, and/or reducing the propensity for the host's cells to be infected by the infectious agent. Thus, in other aspects, provided herein are methods of treating or preventing an infection, e.g., a systemic infection, such as pneumonia, intra-abdominal sepsis, a bacterial infection, and/or a parasitic infection (e.g., malaria). In certain embodiments, the condition may be a viral, bacterial, fungal, or protist infection, and the viral, bacterial, fungal, or protist infection may be resistant to one or more agents for treating the viral, bacterial, fungal, or protist infection. In some embodiments, the bacteria may be gram-negative bacteria.

In yet other aspects, the invention relates to methods of treating or preventing a parasitic worm infection in a subject by conjointly administering to the subject a composition disclosed herein (e.g., a composition comprising hepcidin or mini-hepcidin) and optionally an antiparasitic drug. In some aspects, the invention relates to methods of preventing drug resistance in a subject with a parasitic infection (e.g., malaria or a fluke infection such as fascioliasis), comprising administering to the subject an anti-parasitic drug (e.g., a drug to which a particular infection is resistant) and a composition comprising hepcidin or mini-hepcidin.

In some aspects, the invention relates to methods of treating pneumonia (e.g., bacterial, viral, fungal, and/or parasitic pneumonia) in a subject by conjointly administering to the subject hepcidin or mini-hepcidin and optionally one or more therapeutic drugs for treating the pneumonia. In some embodiments, the invention relates to a method of overcoming, preventing, or inhibiting the development of drug resistance in a subject with pneumonia, comprising conjointly administering to the subject an agent selected from hepcidin and mini-hepcidin and one or more therapeutic drugs, separately or together (e.g., formulated in a single composition).

In certain aspects, the invention relates to a method for treating or preventing malaria in a subject, comprising administering to the subject a composition comprising an antimalarial drug, such as artesunate, and an agent selected from hepcidin and mini-hepcidin. In other aspects, the invention relates to a method of overcoming, preventing or inhibiting the development of, drug resistance in a subject with malaria, comprising administering to the subject a composition comprising an antimalarial drug, such as artesunate, and an agent selected from hepcidin and mini-hepcidin.

The present disclosure also relates to pharmaceutical compositions comprising an antimalarial drug, such as artesunate, and an agent selected from hepcidin and mini-hepcidin. In some aspects, the invention relates to a method for treating or preventing malaria in a subject, comprising administering to the subject a composition comprising an antimalarial drug and an agent selected from hepcidin and mini-hepcidin. In some aspects, the invention relates to a method of overcoming, preventing, or inhibiting the development of drug resistance in a subject with malaria, comprising administering to the subject a composition comprising an antimalarial drug and an agent selected from hepcidin and mini-hepcidin.

### BRIEF DESCRIPTION OF DRAWINGS

FIG. 1 illustrates the change in serum ferritin levels at baseline and 8 days post hepcidin administration in two patients with sickle cell disease and a high ferritin serum baseline.

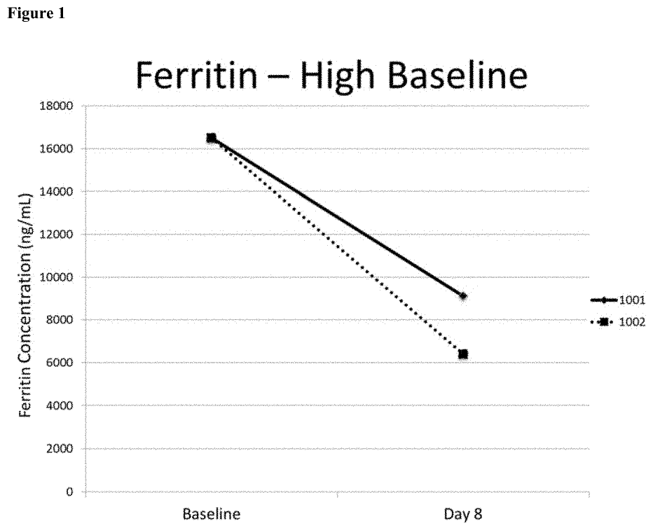

FIG. 2 shows the change in serum ferritin levels at baseline and 8 days post hepcidin administration in patients with sickle cell disease or hereditary hemochromatosis and a normal ferritin serum baseline.

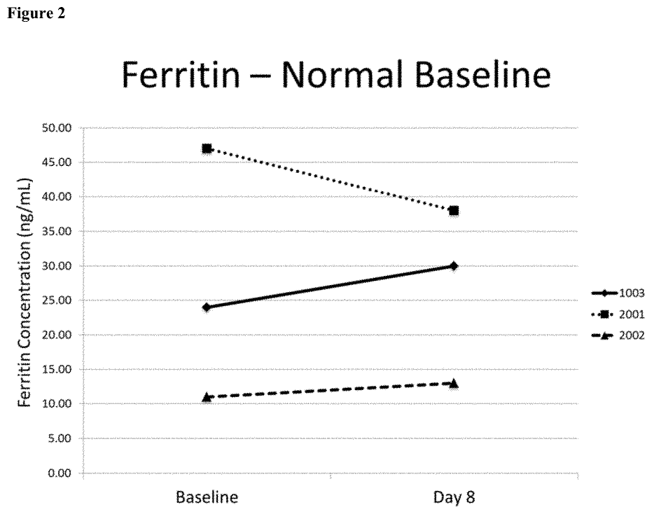

FIG. 3 shows percent change in serum ferritin levels at baseline and 8 days post hepcidin in five patients with either sickle cell disease or hereditary hemochromatosis.

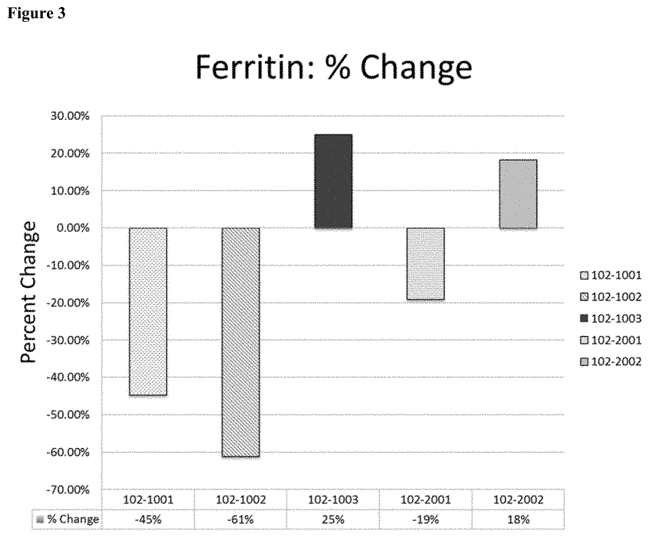

FIG. 4 shows percent of serum transferrin saturation (TSAT) levels at baseline and TSAT at 8 days post hepcidin administration in five patients with either sickle cell disease or hereditary hemochromatosis.

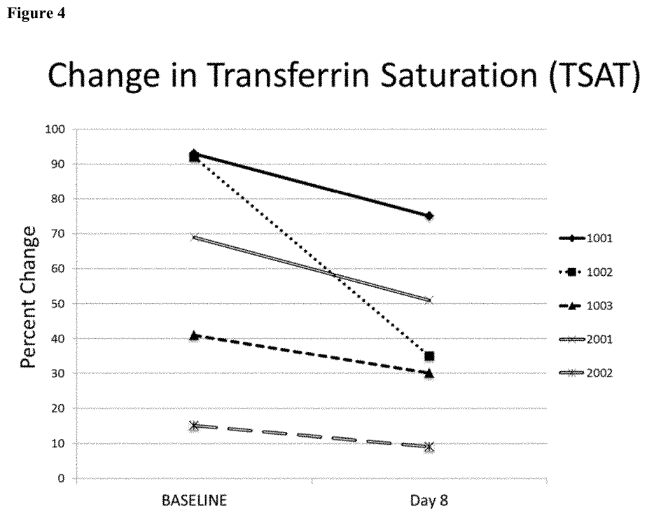

FIG. 5 shows percent change in TSAT levels between baseline and 8 days post hepcidin administration in five patients with either sickle cell disease or hereditary hemochromatosis.

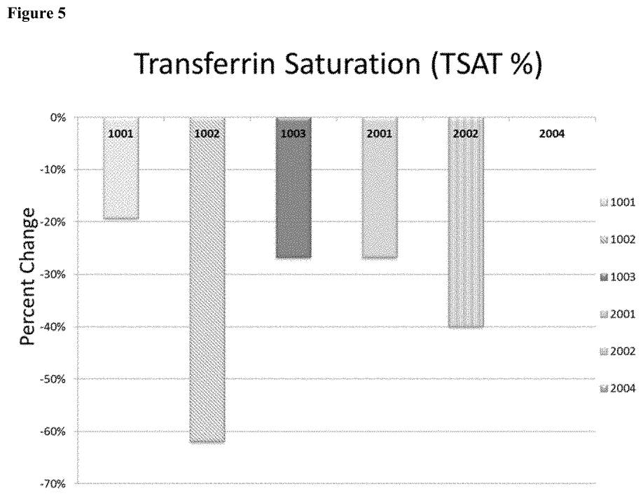

FIG. 6 shows individual serum iron levels in five patients with either sickle cell disease or hereditary hemochromatosis at several time points over an eight day period post hepcidin administration. FIG. 6 also shows the average serum iron levels in a cohort of patients given 1 mg of hepcidin versus a separate cohort of patients given 5 mg hepcidin.

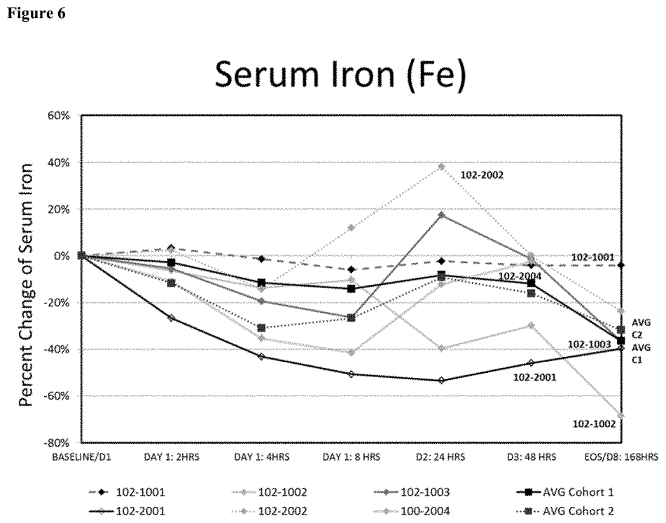

FIG. 7 shows percent change in individual serum iron levels in five patients with either sickle cell disease or hereditary hemochromatosis at several time points over an eight day period post hepcidin administration.

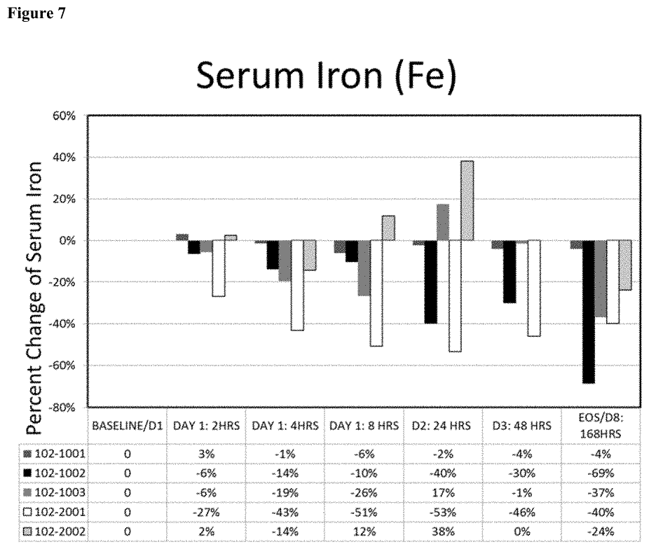

### DETAILED DESCRIPTION

In some aspects, the invention relates to a method for treating a condition in a subject, comprising administering a composition comprising hepcidin or mini-hepcidin to the subject. In some aspects, the invention relates to a method for reducing the serum iron concentration in a subject, comprising administering a composition comprising hepcidin or mini-hepcidin to the subject. The method may comprise administering the composition comprising hepcidin or mini-hepcidin 1, 2, or 3 times per week. Administering hepcidin or mini-hepcidin may comprise subcutaneous administration, such as subcutaneous injection. Alternatively, administering hepcidin or mini-hepcidin may comprise intravenous administration. The subject may have hemochromatosis, α-thalassemia, thalassemia intermedia, β-thalassemia, sickle cell disease, refractory anemia, sideroblastic anemia, Diamond-Blackfan anemia (DBA), or hemolytic anemia. The subject may have a systemic infection, such as pneumonia, intra-abdominal sepsis, a bacterial infection, and/or a parasitic infection (e.g., malaria). In some aspects, the invention relates to a pharmaceutical composition comprising an antimalarial drug, such as artesunate, and an agent selected from hepcidin and mini-hepcidin. In some aspects, the invention relates to a method for treating or preventing malaria in a subject, comprising administering to the subject a composition comprising an antimalarial drug, such as artesunate, and an agent selected from hepcidin and mini-hepcidin. In some aspects, the invention relates to a method of overcoming, preventing or inhibiting the development of, drug resistance in a subject with malaria, comprising administering to the subject a composition comprising an antimalarial drug, such as artesunate, and an agent selected from hepcidin and mini-hepcidin.

### I. Dosing

The method may comprise administering about 10 μg to about 1 gram of hepcidin or mini-hepcidin to the subject, such as about 100 μg to about 100 mg, about 200 μg to about 50 mg, or about 500 μg to about 10 mg, about 500 μg to about 5 mg, or about 500 μg to about 2 mg of hepcidin or mini-hepcidin. The method may comprise administering about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

Administering a composition comprising hepcidin or mini-hepcidin to the subject comprises administering a bolus of the composition.

The method may comprise administering the composition to the subject at least once per month, such as at least once per week. The method may comprise administering the composition to the subject 1, 2, 3, 4, 5, 6, or 7 times per week. In preferred embodiments, the method comprises administering the composition to the subject 1, 2, or 3 times per week.

The method may comprise administering about 10 μg to about 1 gram of hepcidin or mini-hepcidin to the subject each time the composition is administered, such as about 100 μg to about 100 mg, about 200 μg to about 50 mg, about 500 μg to about 10 mg, about 500 μg to about 5 mg, or about 500 μg to about 2 mg of hepcidin or mini-hepcidin. The method may comprise administering about 100 μg, about 150 μg, about 200 μg, about 250 μg, about 300 μg, about 333 μg, about 400 μg, about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin to the subject each time the composition is administered.

In preferred embodiments, less than about 200 mg hepcidin or mini-hepcidin is administered to a human subject each time the composition is administered. In some embodiments, less than about 150 mg hepcidin or mini-hepcidin is administered to a human subject each time the composition is administered, such as less than about 100 mg, less than about 90 mg, less than about 80 mg, less than about 70 mg, less than about 60 mg, or less than about 50 mg.

Surprisingly, doses of hepcidin display efficacy in human subjects at doses of 1 mg hepcidin and 5 mg hepcidin. Efficacy at this dosing was not expected based on animal studies in mice, rats, and dogs. Accordingly, in some embodiments, less than 10 mg of hepcidin or mini-hepcidin is administered to a human subject each time the composition is administered, such as less than about 9 mg, less than about 8 mg, less than about 7 mg, less than about 6 mg, less than about 5 mg, less than about 4 mg, less than about 3 mg, less than about 2 mg, or less than about 1 mg. In some embodiments, about 100 μg to about 10 mg of hepcidin or mini-hepcidin is administered to a human subject each time the composition is administered, such as about 100 μg to about 9 mg, about 100 μg to about 8 mg, about 100 μg to about 7 mg, about 100 μg to about 6 mg, about 100 μg to about 5 mg, about 100 μg to about 4 mg, about 100 μg to about 3 mg, about 100 μg to about 2 mg, or about 100 μg to about 1 mg.

### II. Indications

The condition may be α-thalassemia, thalassemia intermedia, β-thalassemia, hemochromatosis, sickle cell disease, refractory anemia, or hemolytic anemia. The condition may be hemochromatosis and the hemochromatosis may be hereditary hemochromatosis. The condition may be hemochromatosis and the hemochromatosis may be associated with hepatocarcinoma, cardiomyopathy, or diabetes. The condition may be anemia. Anemia may be, for example, a hemoglobinopathy, sideroblastic anemia, anemia associated with myelodysplastic syndrome (MDS), or a congenital anemia. The condition may be myelodysplastic syndrome (MDS). The condition may be Diamond-Blackfan anemia. The condition may be hemoglobinopathy, sideroblastic anemia, or a congenital anemia. In some embodiments, the condition may be hepatocarcinoma, cardiomyopathy, or diabetes.

The condition may be a viral, bacterial, fungal, or protist infection. The condition may be a viral, bacterial, fungal, or protist infection, and the viral, bacterial, fungal, or protist infection may be resistant to one or more agents for treating the viral, bacterial, fungal, or protist infection. In some embodiments, the subject has abdominal sepsis and/or a systemic infection.

In some embodiments, the condition is a viral infection, and the virus is hepatitis B, hepatitis C, or dengue virus.

In some embodiments, the condition is a bacterial infection, and the bacteria is Escherichia coli, Mycobacterium (such as M. africanum, M. avium, M. tuberculosis, M. bovis, M. canetti, M. kansasii, M. leprae, M. lepromatosis, or M. micron), Neisseria cinerea, Neisseria gonorrhoeae, Staphylococcus epidermidis, Staphylococcus aureus, or Streptococcus agalactiae. The bacteria may be gram-negative bacteria, such as Acetic acid bacteria, Acinetobacter Baumann, Bdellovibrio, Borrelia, Bortadella, Burkhoderia, Chlamydia, Cyanobacteria, Campylobacter, Citrobacter, Enterobacter, Neisseria meningitidis, Fusobacterium, green sulfur bacteria, green non-sulfur bacteria, Haemophilus Influenzae, Helicobacter, Hemophilus, Klebsiella, Legionella, Leptospiria, Moraxella, Nitrobacter, Pseudomonas, Proteus, Rickettsia, Serratia, Salmonella, Shigella, spirochaetes, Stenotrophomonas, Thiobacter, Treponema, Vibrio, or Yersinia. The condition may be a bacterial infection and the bacterial infection may be tuberculosis.

In some embodiments, the condition is a fungal infection, and the fungus is Candida albicans. The condition may be a fungal infection, and the fungal infection may be Mucormycosis.

In some embodiments, the condition is a protist infection, and the protist is Trypanosoma cruzi, Plasmodium (such as P. falciparum, P. vivax, P. ovale, or P. malariae), Trypanosoma brucei (such as T. brucei gambiense or T. brucei rhodesiense), or Leishmania. In some embodiments, the protist is Naegleria. The condition may be Chagas disease, malaria, African sleeping sickness, or leishmaniasis.

The method may comprise the conjoint administration of 4-aminosalicylic acid, aldesulfone, amikacin, amithiozone, bedaquiline, capreomycin, clofazimine, cycloserine, dapsone, delamanid, ethambutol, a fluoroquinolone, isoniazid, kanamycin, modified vaccinia Ankara 85A (MVA85A), morinamide, ofloxacin, pyrazinamide, recombinant Bacillus Calmette-Guérin 30 (rBCG30), rifampicin, rifater, streptomycin, terizidone, and/or thioacetazone to the subject. The method may comprise the conjoint administration of balofloxacin, cinoxacin, ciprofloxacin, clinafloxacin, danofloxacin, delafloxacin, difloxacin, enoxacin, enrofloxacin, fleroxacin, Fourth-generation, gatifloxacin, gemifloxacin, grepafloxacin, ibafloxacin, JNJ-Q2, levofloxacin, lomefloxacin, marbofloxacin, moxifloxacin, nadifloxacin, nalidixic acid, nemonoxacin, norfloxacin, ofloxacin, orbifloxacin, oxolinic acid, pazufloxacin, pefloxacin, pipemidic acid, piromidic acid, prulifloxacin, rosoxacin, rufloxacin, sarafloxacin, sitafloxacin, sparfloxacin, temafloxacin, tosufloxacin, and/or trovafloxacin to the subject. In certain such embodiments, the condition may be tuberculosis and/or a Mycobacterium infection. The condition may be tuberculosis and the tuberculosis may be a drug-resistant tuberculosis. The condition may be tuberculosis and the tuberculosis may be multi-drug-resistant tuberculosis (MDR-TB), extensively drug-resistant tuberculosis (XDR-TB), or totally drug-resistant tuberculosis (TDR-TB). The condition may be tuberculosis, and the tuberculosis may not be drug-resistant, multi-drug-resistant, extensively drug-resistant, or totally drug-resistant. The condition may be tuberculosis and/or a Mycobacterium infection and the condition may be resistant to isoniazid, ethambutol, rifampicin, pyrazinamide, ofloxacin, one or more fluoroquinolones, amikacin, kanamycin, and/or capreomycin.

The method may comprise the conjoint administration of fluconazole, ketoconazole, miconazole, and/or itraconazole to the subject. In certain such embodiments, the condition may be Chagas disease and/or Trypanosoma cruzi infection, and the condition may be resistant to one or more of fluconazole, ketoconazole, miconazole, and/or itraconazole. The method may comprise the conjoint administration of fluconazole, benznidazole, and/or amphotericin B to the subject.

The condition may be African sleeping sickness and the method may comprise conjointly administering an arsenical and/or diamidine to the subject. The condition may be African sleeping sickness and/or Trypanosoma bruce infection, and the condition may be resistant to arsenicals and/or diamidines.

The condition may be leishmaniasis and the method may comprise conjointly administering a pentavalent antimonial to the subject. The condition may be leishmaniasis and the condition may be resistant to pentavalent antimonials. The method may comprise conjointly administering amphotericin, amphotericin B, pentavalent antimonials, miltefosine, paromomycin, and/or fluconazole to the subject.

The condition may be malaria. The condition may be malaria and the malaria may be resistant to one or more agents for treating malaria. The condition may be malaria, and the method may comprise conjointly administration of chloroquine, quinine, sulfadoxine-pyrimethamine, halofantrine, atovaquone, and/or mefloquine to the subject. The condition may be malaria, and the malaria may be resistant to one or more of chloroquine, quinine, sulfadoxine-pyrimethamine, halofantrine, atovaquone, and/or mefloquine. The condition may be a multidrug-resistant falciparum malaria infection. The methods provided herein may include treating malaria in a subject with a composition comprising hepcidin or mini-hepcidin in combination with an antimalarial drug. The method may comprise the conjoint administration of one or more antimalarial drugs (e.g., tetracyclines, guanine like drugs, and artemesinin derivatives) to the subject. Exemplary antimalarial drugs include tetracyclines (e.g., tetracycline or tetracycline derivatives), proguanil, chlorproguanil, pyronaridine, lumefantrinel, mefloquine, dapsone, atovaquone, and/or artesunate. The method may comprise the conjoint administration of artemisinin or an artemisinin derivative to the subject. The method may comprise the conjoint administration of artesunate, artemisinin, dihydro-artemisinin, artelinate, arteether, and/or artemether to the subject.

In some aspects, the malaria is a drug-resistant strain of malaria. In some aspects, the methods provided herein are methods of preventing antimalarial drug resistance in a subject by conjointly administering to the subject a composition to induce iron depravation (e.g., a composition comprising hepcidin or mini-hepcidin) in the subject and an antimalarial drug (e.g., an antimalarial drug disclosed herein). The method may comprise the conjoint administration of artemisinin or an artemisinin derivative to the subject. In some aspects, the methods provided herein are methods of preventing artemisinin or artemisinin derivative drug resistance in a subject by administering to the subject a composition comprising hepcidin or mini-hepcidin conjointly with artemisinin or an artemisinin derivative. In some embodiments, provided herein are methods of preventing or treating antimalarial drug resistance in a subject by conjointly administering to the subject a composition comprising hepcidin or mini-hepcidin and an antimalarial drug.

In some aspects, provided herein are methods of treating malaria in a subject by administering a composition comprising hepcidin or mini-hepcidin to the subject. In some embodiments, the subject has been treated for malaria with an antimalarial drug (e.g., an antimalarial drug disclosed herein) prior to administration of a composition comprising hepcidin or mini-hepcidin. In some embodiments, the subject has adverse side effects in response to antimalarial drug treatment. In some aspects, the subject is refractory to antimalarial drugs. In some embodiments, the subject is contraindicated to antimalarial drugs. The subject may have a glucose-6-phosphate dehydrogenase (G6PD) deficiency. G6PD deficiency is a X-chromosomally transmitted disorder that affects red blood cells, which carry oxygen from the lungs to tissues throughout the body. In affected individuals, a defect in glucose-6-phosphate dehydrogenase causes red blood cells to break down prematurely. This destruction of red blood cells is called hemolysis. The most common medical problem associated with glucose-6-phosphate dehydrogenase deficiency is hemolytic anemia, which occurs when red blood cells are destroyed faster than the body can replace them. In people with glucose-6-dehydrogenase deficiency, hemolytic anemia is most often triggered by bacterial or viral infections or by certain drugs (such as medications used to treat malaria). In some aspects, provided herein are methods of treating malaria in a subject by determining whether a subject has a G6PD deficiency, and, if the subject has a G6PD deficiency, administering to the subject a compositions comprising a hepcidin or mini-hepcidin disclosed herein. The composition may be conjointly administered with an antimalarial drug. The subject may be screened for G6PD deficiency by semi-quantitative or quantitative analysis. Semi-quantitative analysis includes tests thsu detect the generation of co-enzyme products produced as a result of G6PD activity, such the generation of nicotinamide adenine dinucleotide phosphate (NADPH) from nicotinamide adenine dinucleotide phosphate (NADP). One example of this test is the fluorescent spot test. This test that measures the generation of NADPH from NADP. The test is positive if the blood spot fails to show fluorescence under ultraviolet light. A variant of the spot test includes a test that can be interpreted by simple color change with a naked eye examination. Other semi-quantitative methods may be employed, including determining NADPH concentration indirectly by, for example, the methaemoglobin reduction test (MRT). This test measures methaemoglobin levels produced after NADPH oxidation. Yet another test that may be employed is a cytochemical typing assay, which provides a fluorometric readout of the classic methaemoglobin reduction test at the level of an individual red blood cell. Quantitative tests include spectrophotometric assays, wherein the rate of NADPH generation is spectrophotometrically measured at a specific wavelength. Other tests for G6PD deficiency include DNA based genotyping and sequencing.

Certain compositions of the present disclosure comprise an antimalarial drug and an agent selected from hepcidin and mini-hepcidin. In some aspects, provided herein are methods of treating or preventing malaria in a subject by administering to the subject a composition comprising an antimalarial drug and an agent selected from hepcidin and mini-hepcidin. In some embodiments, the malaria is a drug-resistant strain of malaria. In some aspects, the methods provided herein may include overcoming, preventing, or inhibiting the development of drug resistance in a subject with malaria with a composition comprising an antimalarial drug and an agent selected from hepcidin and mini-hepcidin. Exemplary antimalarial drugs include tetracyclines (e.g., tetracycline or tetracycline derivatives), proguanil, chlorproguanil, pyronaridine, lumefantrinel, mefloquine, dapsone, atovaquone, artesunate and artemisinin, preferably artesunate. Thus, preferred methods may comprise administering a composition comprising artesunate and an agent selected from hepcidin and mini-hepcidin to the subject.

The condition may be sickle cell disease. In some embodiments, the subject is diagnosed with sickle cell disease or sickle cell anemia. Hepcidin or mini-hepcidin may be administered to the subject at a dose that does not induce a whole-body iron deficiency or worsen an existing iron deficiency in the subject. Iron deficiency may be the result of ineffective erythropoiesis, low levels of serum iron, or a decrease in iron binding capacity. A physician or veterinarian having ordinary skill in the art can readily determine and prescribe the effective amount of the composition (e.g., composition comprising hepcidin or mini-hepcidin) required. For example, the physician or veterinarian could prescribe and/or administer doses of the compounds employed in the composition at levels lower than that required in order to achieve the desired therapeutic effect and gradually increase the dosage until the desired effect is achieved.

The condition may be a parasitic worm infection. Provided herein are methods of treating or preventing a parasitic worm infection in a subject by conjointly administering to the subject an antiparasitic drug and a composition disclosed herein (e.g., a composition comprising hepcidin or mini-hepcidin). The parasitic worm may be a fluke (e.g., a liver fluke such as a Fasciola parasite). The subject may have fascioliasis or paragonimiasis. The anitparasitic drug may be an anthelmintic. In some embodiments, the antiparasitic drug may be triclabendazole, nitazoxanide, metronidazole, niclofolan, chloroquine, artesunate, armamentarium, praziquantel, bithionol, emetine, dehydroemetine, or derivatives thereof. In preferred such embodiments, the antiparasitic drug is triclabendazole. In some embodiments, the subject has a drug-resistant parasitic worm infection (e.g., triclabendazole-resistant fascioliasis).

In some aspects, provided herein are methods of treating or preventing fascioliasis in a subject, comprising conjointly administering to the subject triclabendazole and a composition comprising hepcidin or mini-hepcidin. Also provided herein are methods of preventing drug resistance in a subject with a parasitic infection (e.g., a fluke infection such as fascioliasis), comprising administering to the subject an anti-parasitic drug (e.g., a drug to which a particular infection is resistant) and a composition comprising hepcidin or mini-hepcidin. The anitparasitic drug may be an anthelmintic. In some embodiments, the antiparasitic drug may be triclabendazole, nitazoxanide, metronidazole, niclofolan, chloroquine, artesunate, armamentarium, praziquantel, bithionol, emetine, dehydroemetine, or derivatives thereof.

The condition may be pneumonia. In some embodiments, the subject may have a drug-resistant pneumonia, such as antibiotic-resistant pneumonia. The pneumonia may be bacterial pneumonia, viral pneumonia, fugal pneumonia, or parasitic pneumonia. Provided herein are methods of treating pneumonia (e.g., bacterial pneumonia) in a subject by conjointly administering to the subject one or more antibiotics (e.g., macrolides, ketolides, fluoroketolides, tetracyclines, fluoroquinolones, cephalosporins, penicillins, atovaquone, Trimethoprim-sulfamethoxazole, or vancomycin) and hepcidin or mini-hepcidin. Exemplary methods of treating viral pneumonia may comprise conjointly administering to the subject an antiviral drug (e.g., amantadine, rimantadine, zanamivir, acyclovir, ribavarin, or oseltamivir) and hepcidin or mini-hepcidin. Exemplary methods of treating fungal pneumonia may comprise conjointly administering to the subject an antifungal drug (e.g., amphotericin B, voriconazole, posaconazole, fluconazole, itraconazole, flucytosine, ketoconazole, triazoles, echinocandins, isavuconazole, atovaquone, or caspofungin) and hepcidin or mini-hepcidin. Exemplary methods of treating parasitic pneumonia may comprise conjointly administering to the subject an antiparasitic drug (e.g., an anthelmintic) and hepcidin or mini-hepcidin.

### III. Subjects

The subject may be a mammal. The subject may be a rodent, lagomorph, feline, canine, porcine, ovine, bovine, equine, or primate. In preferred embodiments, the subject is a human. The subject may be a female or male. The subject may be an infant, child, or adult.

In some embodiments, the serum iron concentration of the subject is at least about 50 μg/dL prior to administering the composition, such as at least about 55 μg/dL, at least about 60 μg/dL, at least about 65 μg/dL, at least about 70 μg/dL, at least about 75 μg/dL, at least about 80 μg/dL, at least about 85 μg/dL, at least about 90 μg/dL, at least about 95 μg/dL, at least about 100 μg/dL, at least about 110 μg/dL, at least about 120 μg/dL, at least about 130 μg/dL, at least about 140 μg/dL, at least about 150 μg/dL, at least about 160 μg/dL, at least about 170 μg/dL, at least about 175 μg/dL, at least about 176 μg/dL, at least about 177 μg/dL, at least about 180 μg/dL, at least about 190 μg/dL, at least about 200 μg/dL, at least about 210 μg/dL, at least about 220 μg/dL, at least about 230 μg/dL, at least about 240 μg/dL, at least about 250 μg/dL, at least about 260 μg/dL, at least about 270 μg/dL, at least about 280 μg/dL, at least about 290 μg/dL, or at least about 300 μg/dL. The serum iron concentration of the subject may be about 50 μg/dL to about 500 μg/dL prior to administering the composition, such as about 55 μg/dL to about 500 μg/dL, about 60 μg/dL to about 500 μg/dL, about 65 μg/dL to about 500 μg/dL, about 70 μg/dL to about 500 μg/dL, about 75 μg/dL to about 500 μg/dL, about 80 μg/dL to about 500 μg/dL, about 85 μg/dL to about 500 μg/dL, about 90 μg/dL to about 500 μg/dL, about 95 μg/dL to about 500 μg/dL, about 100 μg/dL to about 500 μg/dL, about 110 μg/dL to about 500 μg/dL, about 120 μg/dL to about 500 μg/dL, about 130 μg/dL to about 500 μg/dL, about 140 μg/dL to about 500 μg/dL, about 150 μg/dL to about 500 μg/dL, about 160 μg/dL to about 500 μg/dL, about 170 μg/dL to about 500 μg/dL, about 175 μg/dL to about 500 μg/dL, about 176 μg/dL to about 500 μg/dL, about 177 μg/dL to about 500 μg/dL, about 180 μg/dL to about 500 μg/dL, about 190 μg/dL to about 500 μg/dL, about 200 μg/dL to about 500 μg/dL, about 210 μg/dL to about 500 μg/dL, about 220 μg/dL to about 500 μg/dL, about 230 μg/dL to about 500 μg/dL, about 240 μg/dL to about 500 μg/dL, about 250 μg/dL to about 500 μg/dL, about 260 μg/dL to about 500 μg/dL, about 270 μg/dL to about 500 μg/dL, about 280 μg/dL to about 500 μg/dL, about 290 μg/dL to about 500 μg/dL, or about 300 μg/dL to about 500 μg/dL.

In preferred embodiments, administering the composition to a subject decreases the serum iron concentration of the subject. For example, administering the composition may decrease the serum iron concentration of a subject by at least about 5 μg/dL, at least about 10 μg/dL, at least about 5 μg/dL, at least about 20 μg/dL, at least about 30 μg/dL, at least about 40 μg/dL, at least about 50 μg/dL, at least about 60 μg/dL, at least about 70 μg/dL, at least about 80 μg/dL, at least about 90 μg/dL, or at least about 100 μg/dL. Administering the composition may decrease the serum iron concentration of the subject for at least 24 hours. For example, administering the composition may decrease the serum iron concentration of the subject by at least about 5 μg/dL for a period of time of at least 24 hours. Administering the composition may decrease the serum iron concentration of the subject by at least about 5 μg/dL for at least 4 hours, at least 6 hours, or at least 12 hours. Administering the composition may decrease the serum iron concentration of the subject by at least about 5 μg/dL for at least 1 day, at least 2 days, at least 3 days, at least 4 days, at least 5 days, at least 6 days, at least 7 days, or at least 8 days. Administering the composition may decrease the serum iron concentration of the subject by at least about 5%, such as at least about 10%, at least about 15%, at least about 20%, at least about 25%, or even at least about 30%. Administering the composition may decrease the serum iron concentration of the subject by at least about 5% for at least 4 hours, at least 6 hours, or at least 12 hours. Administering the composition may decrease the serum iron concentration of the subject by at least about 5% for at least 1 day, at least 2 days, at least 3 days, at least 4 days, at least 5 days, at least 6 days, at least 7 days, or at least 8 days.

In some embodiments, the subject has a serum hepcidin concentration of less than about 1000 ng/mL prior to administering the composition, such as less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum hepcidin concentration of about 1 ng/mL to about 1000 ng/mL prior to administering the composition, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, the subject has a serum ferritin concentration greater than about 10 ng/mL prior to administering the composition, such as greater than about 20 ng/mL, greater than about 30 ng/mL, greater than about 40 ng/mL, greater than about 50 ng/mL, greater than about 60 ng/mL, greater than about 70 ng/mL, greater than about 80 ng/mL, greater than about 90 ng/mL, greater than about 100 ng/mL, greater than about 200 ng/mL, greater than about 300 ng/mL, greater than about 400 ng/mL, greater than about 500 ng/mL, greater than about 600 ng/mL, greater than about 700 ng/mL, greater than about 800 ng/mL, greater than about 900 ng/mL, greater than about 1000 ng/mL, greater than about 2000 ng/mL, greater than about 3000 ng/mL, greater than about 4000 ng/mL, greater than about 5000 ng/mL, greater than about 6000 ng/mL, greater than about 7000 ng/mL, greater than about 8000 ng/mL, greater than about 9000 ng/mL, or even greater than about 10 μg/mL. The subject may have a serum ferritin concentration of about 10 ng/mL to about 100 μg/mL prior to administering the composition, such as about 20 ng/mL to about 100 μg/mL, about 30 ng/mL to about 100 μg/mL, about 40 ng/mL to about 100 μg/mL, about 50 ng/mL to about 100 μg/mL, about 60 ng/mL to about 100 μg/mL, about 70 ng/mL to about 100 μg/mL, about 80 ng/mL to about 100 μg/mL, about 90 ng/mL to about 100 μg/mL, about 100 ng/mL to about 100 μg/mL, about 200 ng/mL to about 100 μg/mL, about 300 ng/mL to about 100 μg/mL, about 400 ng/mL to about 100 μg/mL, about 500 ng/mL to about 100 μg/mL, about 600 ng/mL to about 100 μg/mL, about 700 ng/mL to about 100 μg/mL, about 800 ng/mL to about 100 μg/mL, about 900 ng/mL to about 100 μg/mL, or about 1000 ng/mL to about 100 μg/mL. The subject may have a serum ferritin concentration of about 10 ng/mL to about 20 μg/mL prior to administering the composition, such as about 20 ng/mL to about 20 μg/mL, about 30 ng/mL to about 20 μg/mL, about 40 ng/mL to about 20 μg/mL, about 50 ng/mL to about 20 μg/mL, about 60 ng/mL to about 20 μg/mL, about 70 ng/mL to about 20 μg/mL, about 80 ng/mL to about 20 μg/mL, about 90 ng/mL to about 20 μg/mL, about 100 ng/mL to about 20 μg/mL, about 200 ng/mL to about 20 μg/mL, about 300 ng/mL to about 20 μg/mL, about 400 ng/mL to about 20 μg/mL, about 500 ng/mL to about 20 μg/mL, about 600 ng/mL to about 20 μg/mL, about 700 ng/mL to about 20 μg/mL, about 800 ng/mL to about 20 μg/mL, about 900 ng/mL to about 20 μg/mL, or about 1000 ng/mL to about 20 μg/mL.

In some embodiments, the subject has a serum ferritin concentration of less than about 10 μg/mL prior to administering the composition, such as less than about 1000 ng/mL, less than about 900 ng/mL, less than about 800 ng/mL, less than about 700 ng/mL, less than about 600 ng/mL, less than about 500 ng/mL, less than about 400 ng/mL, less than about 300 ng/mL, less than about 200 ng/mL, less than about 100 ng/mL, less than about 90 ng/mL, less than about 80 ng/mL, less than about 70 ng/mL, less than about 60 ng/mL, less than about 50 ng/mL, less than about 40 ng/mL, less than about 30 ng/mL, less than about 20 ng/mL, or less than about 10 ng/mL. The subject may have a serum ferritin concentration of about 1 ng/mL to about 1000 ng/mL prior to administering the composition, such as about 1 ng/mL to about 900 ng/mL, about 1 ng/mL to about 800 ng/mL, about 1 ng/mL to about 700 ng/mL, about 1 ng/mL to about 600 ng/mL, about 1 ng/mL to about 500 ng/mL, about 1 ng/mL to about 400 ng/mL, about 1 ng/mL to about 300 ng/mL, about 1 ng/mL to about 200 ng/mL, about 1 ng/mL to about 100 ng/mL, about 1 ng/mL to about 90 ng/mL, about 1 ng/mL to about 80 ng/mL, about 1 ng/mL to about 70 ng/mL, about 1 ng/mL to about 60 ng/mL, about 1 ng/mL to about 50 ng/mL, about 1 ng/mL to about 40 ng/mL, about 1 ng/mL to about 30 ng/mL, about 1 ng/mL to about 20 ng/mL, or about 1 ng/mL to about 10 ng/mL.

In some embodiments, administering the composition decreases the serum ferritin concentration of the subject. For example, administering the composition may decrease the serum ferritin concentration of the subject by at least about 10 ng/mL, at least about 20 ng/mL, at least about 30 ng/mL, at least about 40 ng/mL, at least about 50 ng/mL, at least about 60 ng/mL, at least about 70 ng/mL, at least about 80 ng/mL, at least about 90 ng/mL, or at least about 100 ng/mL.

In some embodiments, the subject has a total body iron content of about 40 to about 50 mg/kg prior to administering the composition. The subject may have a total body iron content greater than about 50 mg/kg prior to administering the composition, such as greater than about 55 mg/kg, greater than about 60 mg/kg, greater than about 65 mg/kg, or greater than about 70 mg/kg.

In some embodiments, the subject has a transferrin saturation percentage greater than about 10% prior to administering the composition, such as greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, or even greater than about 90%. The subject may have a transferrin saturation percentage of about 10% to about 99% prior to administering the composition, such as about 15% to about 99%, about 20% to about 99%, about 25% to about 99%, about 30% to about 99%, about 35% to about 99%, about 40% to about 99%, about 45% to about 99%, about 50% to about 99%, about 55% to about 99%, about 60% to about 99%, about 65% to about 99%, about 70% to about 99%, about 75% to about 99%, about 80% to about 99%, or about 85% to about 99%. The subject may have a transferrin saturation percentage of about 10% to about 95% prior to administering the composition, such as about 15% to about 95%, about 20% to about 95%, about 25% to about 95%, about 30% to about 95%, about 35% to about 95%, about 40% to about 95%, about 45% to about 95%, about 50% to about 95%, about 55% to about 95%, about 60% to about 95%, about 65% to about 95%, about 70% to about 95%, about 75% to about 95%, about 80% to about 95%, or about 85% to about 95%.

In some embodiments, administering the composition decreases the transferrin saturation percentage of the subject. For example, administering the composition to a subject may decrease the transferrin saturation percentage of the subject by at least about 1% transferrin saturation, such as at least about 2% transferrin saturation, at least about 3% transferrin saturation, at least about 4% transferrin saturation, at least about 5% transferrin saturation, at least about 6% transferrin saturation, at least about 7% transferrin saturation, at least about 8% transferrin saturation, at least about 9% transferrin saturation, at least about 10% transferrin saturation, at least about 11% transferrin saturation, at least about 12% transferrin saturation, at least about 13% transferrin saturation, at least about 14% transferrin saturation, at least about 15% transferrin saturation, at least about 16% transferrin saturation, at least about 17% transferrin saturation, at least about 18% transferrin saturation, at least about 19% transferrin saturation, at least about 20% transferrin saturation, at least about 25% transferrin saturation, at least about 30% transferrin saturation, at least about 35% transferrin saturation, at least about 40% transferrin saturation, at least about 45% transferrin saturation, or at least about 50% transferrin saturation.

### IV. Active Agent

The hepcidin peptide is a 25-amino acid peptide with the amino acid sequence set forth in SEQ ID NO:1. The hepcidin peptide is a cleavage product of a larger protein, and the cell membrane protein furin can convert an extracellular hepcidin precursor protein into the hepcidin peptide. The term “hepcidin” as used herein may therefore refer to a peptide comprising the sequence set forth in SEQ ID NO:1, including peptides that are longer than 25 amino acids, such as peptides consisting of 26 to 100 amino acids. Conservative amino acid substitutions, additions, and deletions may be made to SEQ ID NO:1 without significantly affecting the function of hepcidin. Thus, the term “hepcidin” may refer to a peptide comprising an amino acid sequence having at least 90%, 91%, 92%, 93%, 94%, 95%, or 96% sequence homology with the amino acid sequence set forth in SEQ ID NO:1. Sequence homology may be determined using any suitable sequence alignment program, such as Protein Blast (blastp) or Clustal (e.g., ClustalV, ClustalW, ClustalX, or Clustal Omega), e.g., using default parameters, such as default weights for gap openings and gap extensions. Sequence homology may refer to sequence identity. The term “hepcidin” may refer to a peptide comprising an amino acid sequence that is identical to the sequence set forth in SEQ ID NO:1 except that 1, 2, 3, 4, 5, 6, 7, 8, 9, or 10 amino acids of SEQ ID NO:1 are substituted with different amino acids. In preferred embodiments, hepcidin comprises a cysteine at each of the positions in which a cysteine occurs in SEQ ID NO:1.

SEQ ID NO: 1
DTHFPICIFCCGCCHRSKCGMCCKT

N-terminal and C-terminal residues may be deleted from the hepcidin peptide without significantly affecting its function. Thus, in some embodiments, hepcidin refers to a peptide comprising the sequence set forth in SEQ ID NO:2, SEQ ID NO:3, or SEQ ID NO:4, or a peptide comprising an amino acid sequence having at least 90%, 91%, 92%, 93%, 94%, 95%, or 96% sequence homology with the amino acid sequence set forth in SEQ ID NO:2, SEQ ID NO:3, SEQ ID NO:4, or SEQ ID NO:5. The term hepcidin may refer to a peptide comprising an amino acid sequence that is identical to the sequence set forth in SEQ ID NO:2, SEQ ID NO:3, SEQ ID NO:4, or SEQ ID NO:5 except that 1, 2, 3, 4, 5, 6, 7, 8, 9, or 10 amino acids of SEQ ID NO:2, SEQ ID NO:3, SEQ ID NO:4, or SEQ ID NO:5 are substituted with different amino acids. In preferred embodiments, hepcidin comprises a cysteine at each of the positions in which a cysteine occurs in SEQ ID NO:2, SEQ ID NO:3, SEQ ID NO:4, or SEQ ID NO:5.

SEQ ID NO: 2
PICIFCCGCCHRSKCGMCCKT
SEQ ID NO: 3
PICIFCCGCCHRSKCGMCC
SEQ ID NO: 4
ICIFCCGCCHRSKCGMCCKT
SEQ ID NO: 5
CIFCCGCCHRSKCGMCC

In some embodiments, the term “hepcidin” refers to a peptide comprising an amino acid sequence that is identical to the sequence set forth in SEQ ID NO:6, SEQ ID NO:7, SEQ ID NO:8, SEQ ID NO:9, or SEQ ID NO:10. In SEQ ID NO:6, SEQ ID NO:7, SEQ ID NO:8, SEQ ID NO:9, or SEQ ID NO:10, the amino acids labeled “X” may be any amino acid, including naturally occurring and non-naturally occurring amino acids. In some embodiments, each of the amino acids labeled “X” is a naturally occurring amino acid.

SEQ ID NO: 6
XXHXPXCXXCCGCCHRSKCGMCCXX
SEQ ID NO: 7
PXCXXCCGCCHRSKCGMCCKX
SEQ ID NO: 8
PXCXXCCGCCHRSKCGMCC
SEQ ID NO: 9
XCXXCCGCCHRXXCGXCCKX
SEQ ID NO: 10
CXXCCGCCHRXXCGXCC

In preferred embodiments, hepcidin is a molecule that specifically binds to ferroportin and/or iron (e.g., an iron cation). Hepcidin may comprise 1, 2, 3, or 4 disulfide bonds. In preferred embodiments, hepcidin comprises four disulfide bonds. In preferred embodiments, each of the four disulfide bonds is an intramolecular disulfide bond. In preferred embodiments, each of the eight cysteines of SEQ ID NO:1, 2, 3, 4, 5, 6, 7, 8, 9, or 10 participates in one of four intramolecular disulfide bonds with another one of the eight cysteines.

In preferred embodiments, hepcidin has about 10% to 1000% of the activity of a 25 amino acid long peptide comprising the amino acid sequence set forth in SEQ ID NO:1, i.e., wherein the 25 amino acid long peptide comprises the four intramolecular disulfide bonds found in native human hepcidin. For example, hepcidin may have about 50% to about 200% of the activity of a 25 amino acid long peptide comprising the amino acid sequence set forth in SEQ ID NO:1 (i.e., wherein the 25 amino acid long peptide comprises the four intramolecular disulfide bonds found in native human hepcidin), such as about 75% to about 150% of the activity, about 80% to about 120% of the activity, about 90% to about 110% of the activity, or about 95% to about 105% of the activity. The term “activity” may refer to the ability of hepcidin to specifically bind to ferroportin, e.g., thereby inhibiting the transport of intracellular iron into the extracellular space, inhibiting the absorption of dietary iron, and/or reducing serum iron concentration. Activity may refer to the ability of hepcidin to inhibit the transport of intracellular iron into the extracellular space. Activity may refer to the ability of hepcidin to inhibit the absorption of dietary iron. Activity may refer to the ability of hepcidin to reduce serum iron concentration in vivo.

In some embodiments, mini-hepcidin may refer to a mini-hepcidin, modified hepcidin or a hepcidin mimetic peptide. For the purposes of this application, the terms mini-hepcidin, a modified hepcidin, or a hepcidin mimetic peptide may be used interchangeably. Mini-hepcidins, a modified hepcidin, and hepcidin mimetic peptides are disclosed in U.S. Pat. Nos. 9,315,545, 9,328,140, and 8,435,941, each of which are hereby incorporated by reference, in particular for their disclosure of compounds that share one or more activities with hepcidin.

A mini-hepcidin may have the structure of Formula I, or a pharmaceutically acceptable salt thereof:

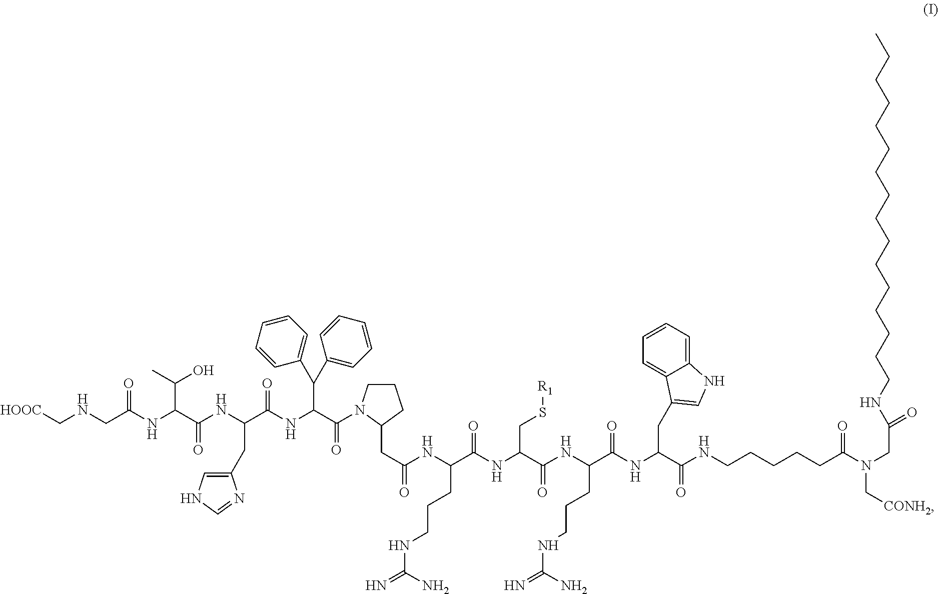

wherein R₁ is, —S—Z₁; —Z₂, —SH, —C(═O)—Z₃ or —S—C(═O)—)—Z₃,
Z₁ is substituted or unsubstituted C₁-C₁₈ alkyl or C₁-C₁₈ alkenyl, wherein the C₁-C₁₈ alkyl or C₁-C₁₈ alkenyl is branched or unbranched or Z₁ is an electron withdrawing or donating group;
Z₂ is substituted or unsubstituted C₁-C₁₈ alkyl or C₁-C₁₈ alkenyl, wherein the C₁-C₁₈ alkyl or C₁-C₁₈ alkenyl is branched or unbranched or Z₂ is an electron withdrawing or donating group;
Z₃ is substituted or unsubstituted C₁-C₁₈ alkyl or C₁-C₁₈ alkenyl, wherein the C₁-C₁₈ alkyl or C₁-C₁₈ alkenyl is branched or unbranched or Z₃ is an electron withdrawing or donating group.

A mini-hepcidin may have the structure of any one of Formulas II-IV, or a pharmaceutically acceptable salt thereof:

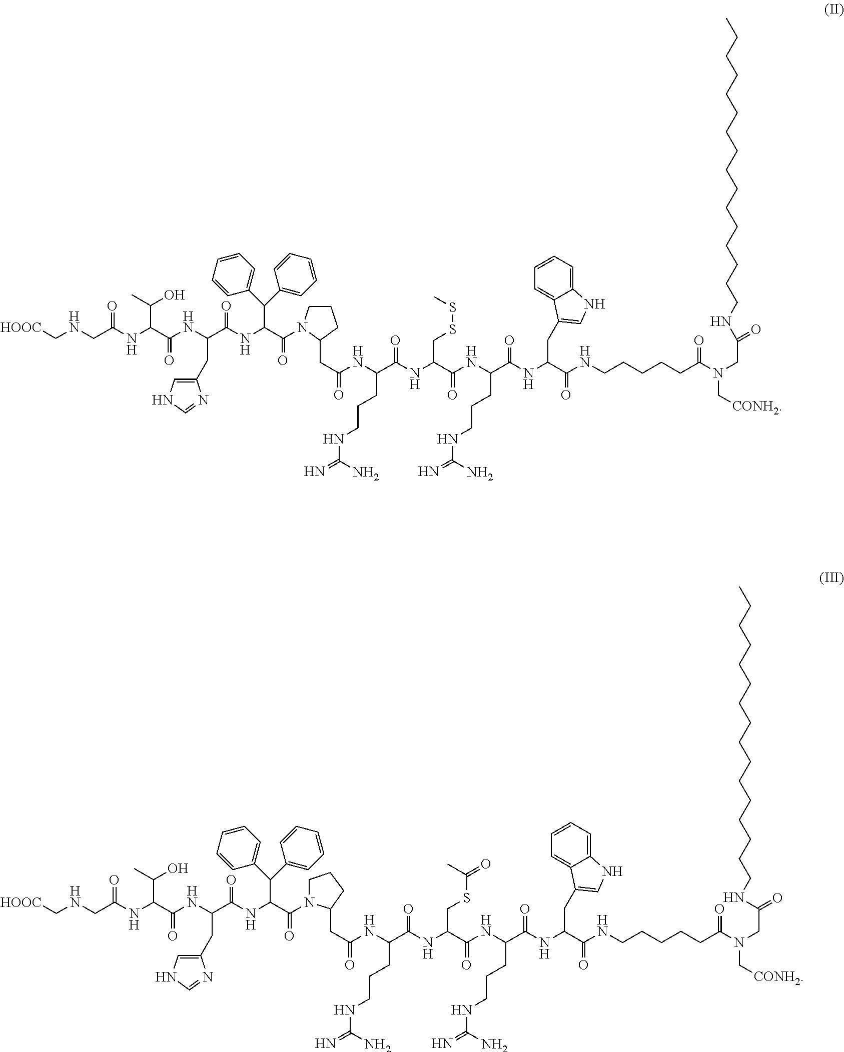 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1 ; CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSC(C)=O)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

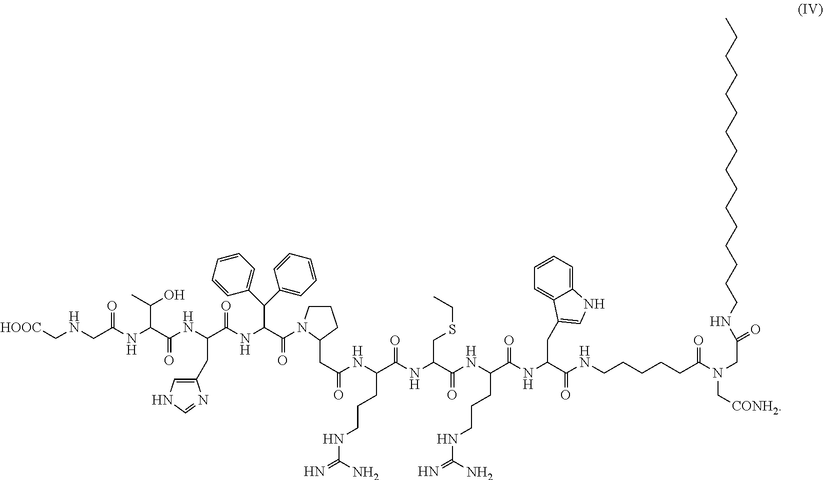 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSCC)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of Formula V, or a pharmaceutically acceptable salt thereof:

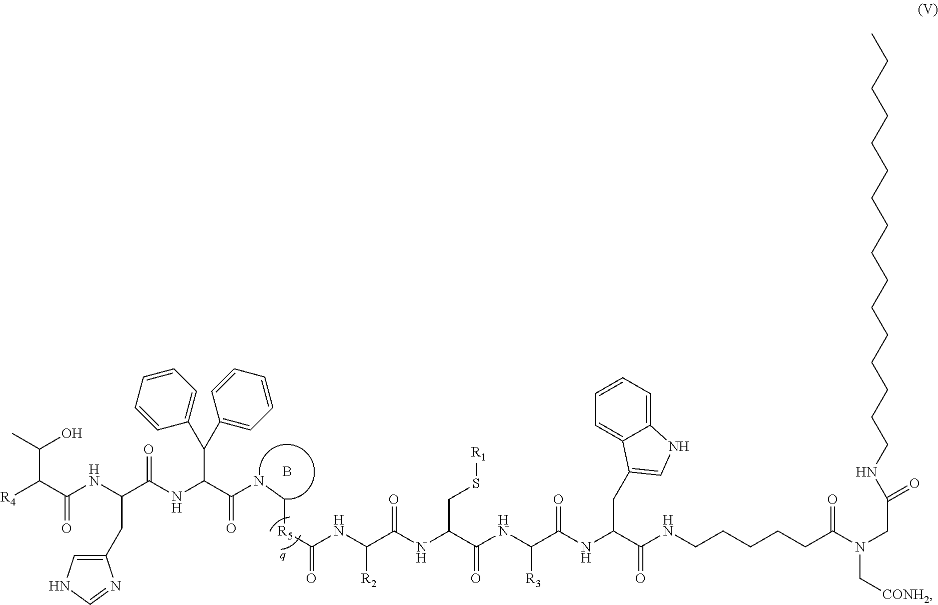

wherein:
R₁ is, H, —S—Z₁; —Z₂, —SH, —C(═O)—Z₃, or —S—C(═O)—Z₃,
R₂ and R₃ are each, independently, optionally substituted C₄-C₇ alkyl,

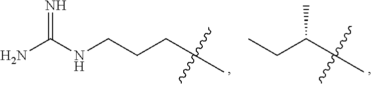

D-Arg, D-Ile, Leu, D-Leu, Thr, D-Thr, Lys, D-Lys, Val, D-Val, D-Nω,ω-dimethyl-arginine, L-Nω,ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
R₅ is CR₆R₇, aryl or heteroaryl;
B is absent or forms a 5-7 membered ring; and
q is 0-6, wherein when R₅ aryl or heteroaryl q is 1 and B is absent;
Z₁ is substituted or unsubstituted C₁-C₁₈ alkyl, wherein the C₁-C₁₈ alkyl is branched or unbranched;
Z₂ is substituted or unsubstituted C₁-C₁₈ alkyl, wherein the C₁-C₁₈ alkyl is branched or unbranched;
Z₃ is substituted or unsubstituted C₁-C₁₈ alkyl, wherein the C₁-C₁₈ alkyl is branched or unbranched;
R₆ and R₇ are each, independently, H, halo, optionally substituted C₁-C₃ alkyl, or haloalkyl, provided that when R₁ is H, the compound does not have the structure of Formula XVI.

A mini-hepcidin may have the structure of any one of Formulas VI-VIII, or a pharmaceutically acceptable salt thereof:

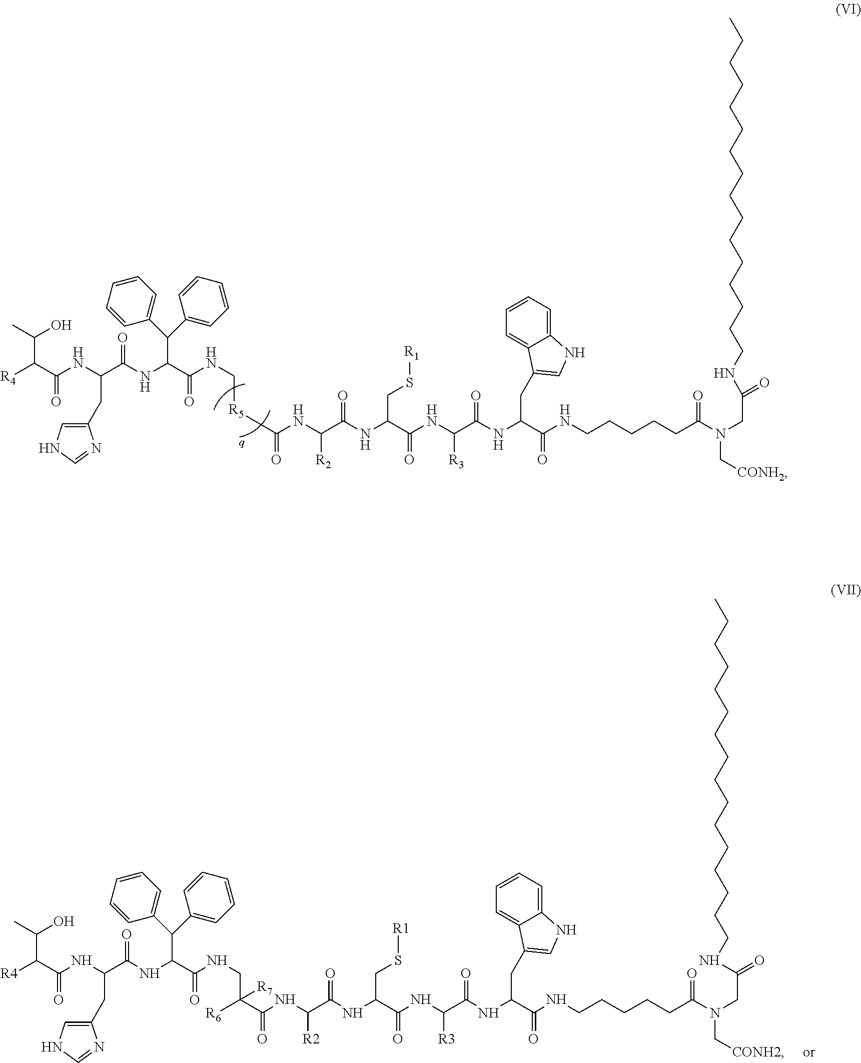

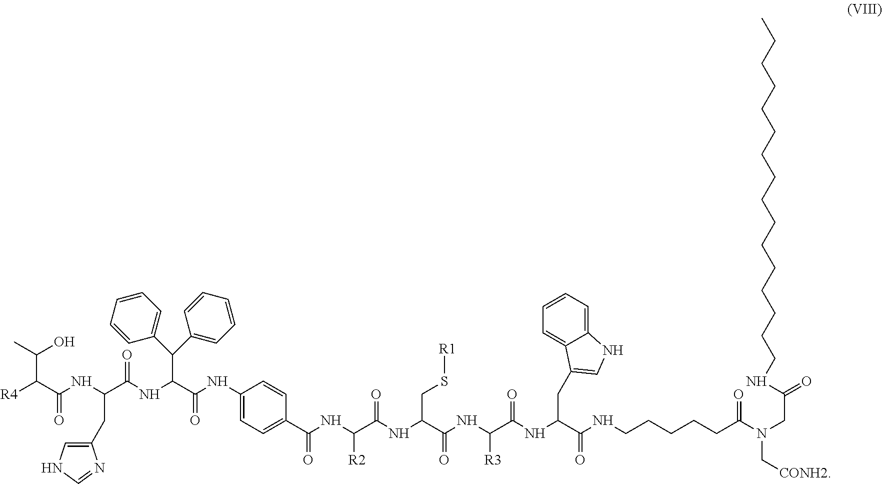

wherein the variables are defined as for Formula V.

A mini-hepcidin may have the structure of Formula IX, or a pharmaceutically acceptable salt thereof:

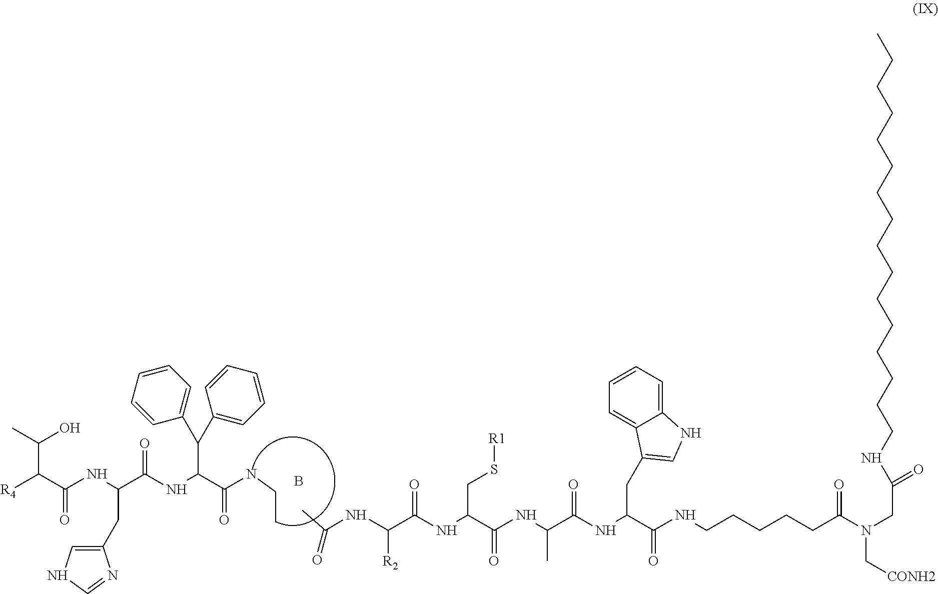

wherein R₁ is H, —S—Z₁, —Z₂, —SH, —S—C(═O)—Z₃, or —C(═O)—Z₃,
R₂ and R₃ are each, independently, optionally substituted C₄-C₇ alkyl,

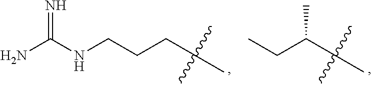

D-Arg, D-Ile, Leu, D-Leu, Thr, D-Thr, Lys, D-Lys, Val, D-Val, D-Nω,ω-dimethyl-arginine, L-Nω,ω-dimethyl-arginine, D-homoarginine, L-homoarginine, D-norarginine, L-norarginine, citrulline, a modified Arg wherein the guanidinium group is modified or substituted, norleucine, norvaline, beta homo-Ile, 1-aminocyclohexane-1-carboxylic acid, N-Me-Arg, N-Me-Ile;
R₄ is Ida, Asp, Acetyl-Asp, (methylamino)pentanedioic acid, Acetyl-Gly-Ida, or Acetyl-Gly-Asp or a derivative thereof to remove its negative charge above pH 4;
B is absent or forms a 5-7 membered ring;
Z₁ is substituted or unsubstituted C₁-C₁₈ alkyl, wherein the C₁-C₁₈ alkyl is branched or unbranched;
Z₂ is substituted or unsubstituted C₁-C₁₈ alkyl, wherein the C₁-C₁₈ alkyl is branched or unbranched; and
Z₃ is substituted or unsubstituted C₁-C₁₈ alkyl, wherein the C₁-C₁₈ alkyl is branched or unbranched;
provided that when R₁ is H, the compound does not have the structure of Formula XVI.

A mini-hepcidin may have the structure of Formula X, or a pharmaceutically acceptable salt thereof:

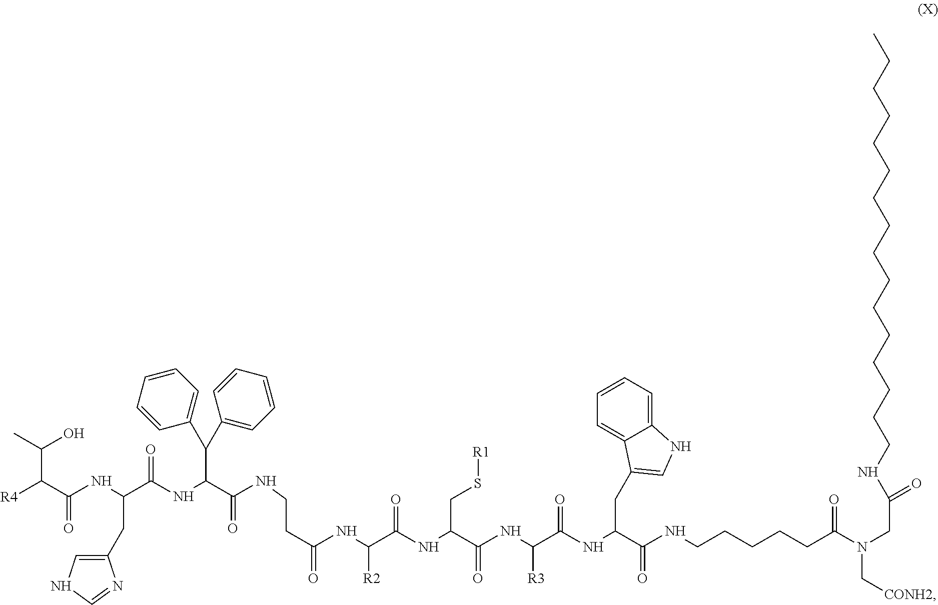

wherein the variables are defined as for Formula IX.

A mini-hepcidin may have the structure of Formula XI, or a pharmaceutically acceptable salt thereof:

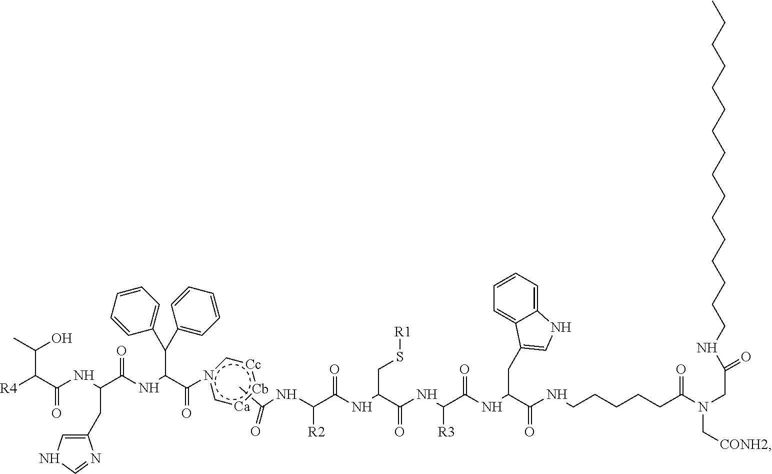

wherein the carbonyl forms a bond with the 6-membered ring at Cₐ, C_{b}, or C_{c} and with the variables as defined for Formula IX.

A mini-hepcidin may have the structure of Formula XII, or a pharmaceutically acceptable salt thereof:

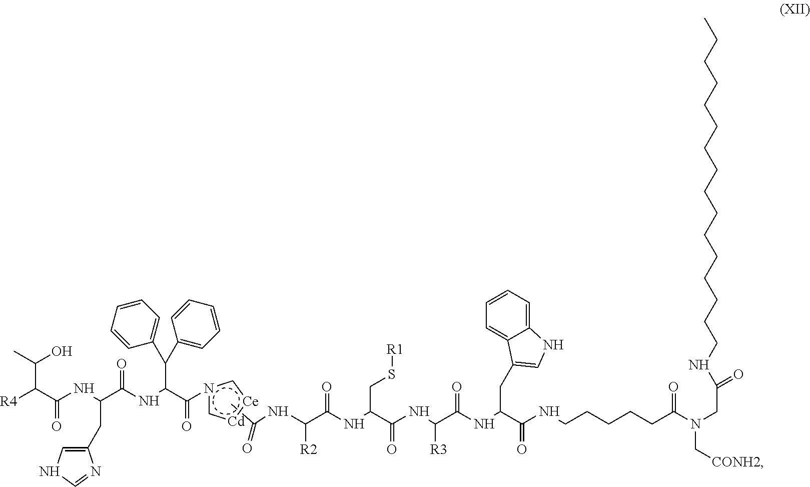

wherein the carbonyl forms a bond with the 5-membered ring at C_{d} or Cₑ, and with the variables as defined for Formula IX.

A mini-hepcidin may have the structure of Formula XIII, or a pharmaceutically acceptable salt thereof:

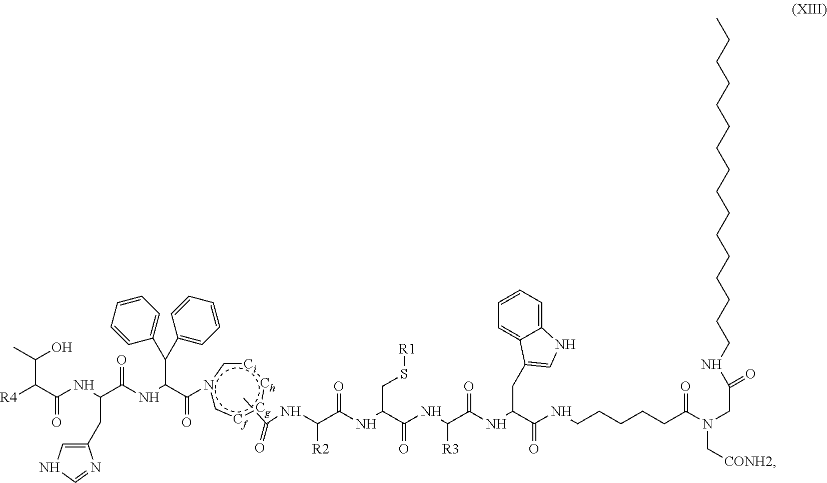

wherein the bond from the carbonyl forms a bond with the 7-membered ring at C_{f}, C_{g}, Cₕ, or Cᵢ and with the variables as defined for Formula IX.

A mini-hepcidin may have the structure of Formula XIV, or a pharmaceutically acceptable salt thereof:

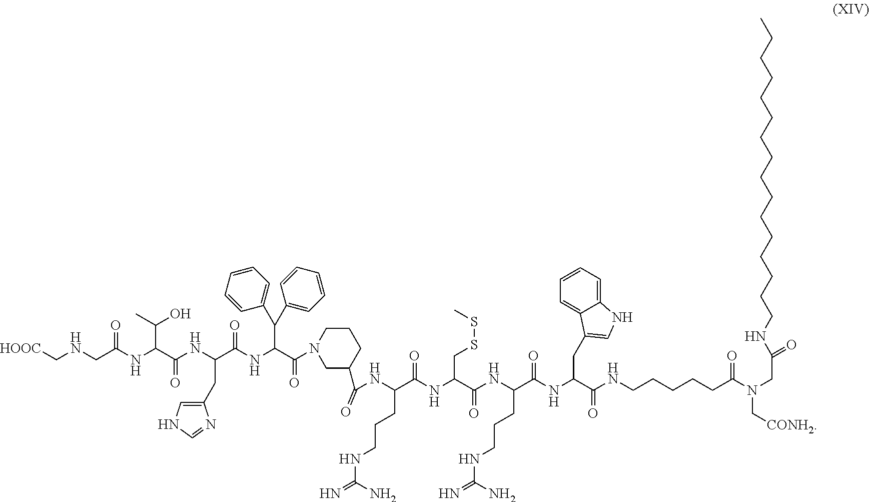 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CSSC)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula XV, or a pharmaceutically acceptable salt thereof:

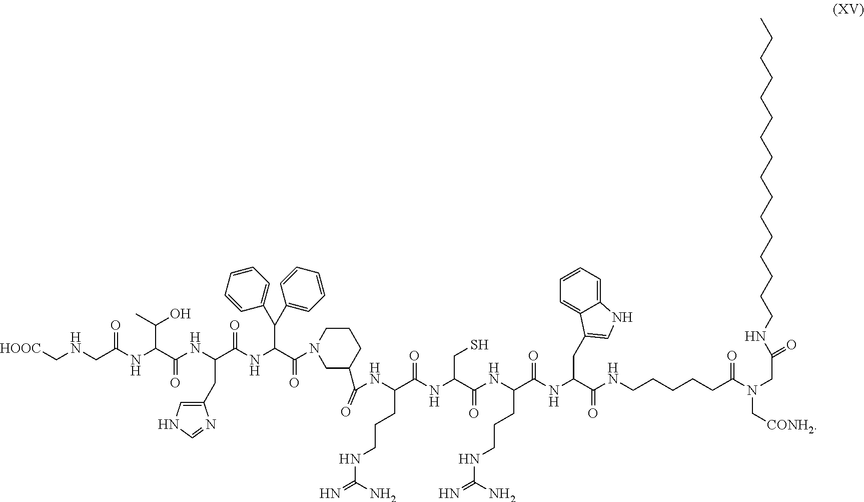 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)C1CCCN(C(=O)C(NC(=O)C(Cc2c[nH]cn2)NC(=O)C(NC(=O)CNCC(=O)O)C(C)O)C(c2ccccc2)c2ccccc2)C1]

A mini-hepcidin may have the structure of Formula P₁-P₂-P₃-P₄-P₅-P₆-P₇-P₈-P₉-P₁₀ or P₁₀-P₉-P₈-P₇-P₆-P₅-P₄-P₃-P₂-P_{1,} or a pharmaceutically acceptable salt thereof, wherein P₁ to P₁₀ are as defined in table 1; X₃ is aminohexanoic acid-Ida(NH-PAL)-NH₂, Ida is iminodiacetic acid; Dpa is 3,3-diphenyl-L-alanine; bhPro is beta-homoproline; Npc is L-nipecotic acid; isoNpc is isonipecotic acid; and bAla is beta-alanine.

TABLE 1

| P₁ | P₂ | P₃ | P₄ | P₅ | P₆ | P₇ | P₈ | P₉ | P₁₀ |
|---|---|---|---|---|---|---|---|---|---|
| Ida | Thr | His | Dpa | bhPro | Arg | Cys-S—CH₃ | Arg | Trp | X₃ |
| Ida | Thr | His | Dpa | bhPro | Arg | Cys-C(═O)CH₃ | Arg | Trp | X₃ |
| Ida | Thr | His | Dpa | bhPro | Arg | Cys-CH₂—CH₃ | Arg | Trp | X₃ |
| Ida | Thr | His | Dpa | Npc | Arg | Cys-S—CH₃ | Arg | Trp | X₃ |
| Ida | Thr | His | Dpa | Npc | Arg | Cys | Arg | Trp | X₃ |
| Ida | Thr | His | Dpa | D-Npc | Arg | Cys-S—CH₃ | Arg | Trp | X₃ |
| Ida | Thr | His | Dpa | isoNpc | Arg | Cys-S—CH₃ | Arg | Trp | X₃ |
| Acetyl-Gly-Ida | Thr | His | Dpa | bhPro | Arg | Cys-S—CH₃ | Arg | Trp | X₃ |
| Ida | Thr | His | Dpa | bAla | Arg | Cys-S—CH₃ | Arg | Trp | X₃ |

A mini-hepcidin may have the structure of Formula XVI, or a pharmaceutically acceptable salt thereof:

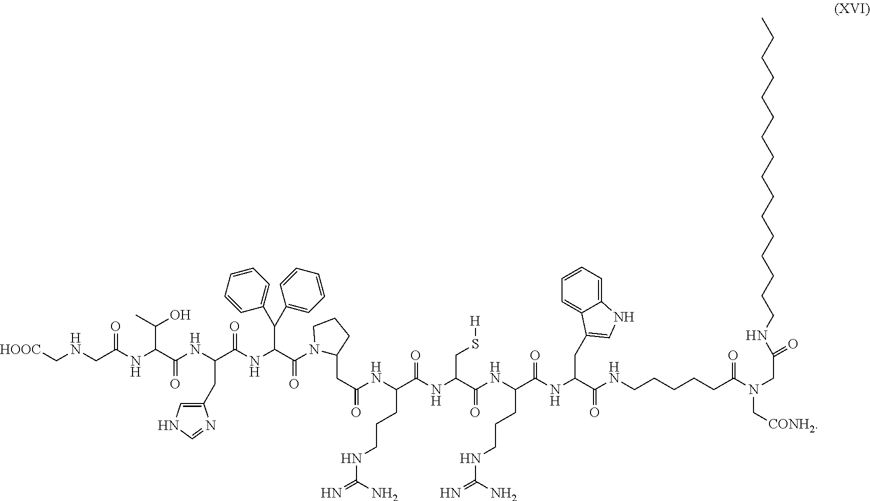 [SMILES: CCCCCCCCCCCCCCCCNC(=O)CN(CC(N)=O)C(=O)CCCCCNC(=O)C(Cc1c[nH]c2ccccc12)NC(=O)C(CCCNC(=N)N)NC(=O)C(CS)NC(=O)C(CCCNC(=N)N)NC(=O)CC1CCCN1C(=O)C(NC(=O)C(Cc1c[nH]cn1)NC(=O)C(NC(=O)CNCC(=O)O)C(C)O)C(c1ccccc1)c1ccccc1]

A mini-hepcidin may have the structure of formula A1-A2-A3-A4-A5-A6-A7-A8-A9-A10, A10-A9-A8-A7-A6-A5-A4-A3-A2-A1, or a pharmaceutically acceptable salt thereof, wherein:
A₁ is L-Asp, L-Glu, pyroglutamate, L-Gln, L-Asn, D-Asp, D-Glu, D-pyroglutamate, D-Gln, D-Asn, 3-aminopentanedioic acid, 2,2′-azanediyldiacetic acid, (methylamino)pentanedioic acid, L-Ala, D-Ala, L-Cys, D-Cys, L-Phe, D-Phe, L-Asp, D-Asp, 3,3-diphenyl-L-alanine, 3,3-diphenyl-D-alanine; and if Al is L-Asp or D-Asp, then A2 is L-Cys or D-Cys; if A1 is L-Phe or D-Phe, then the N-terminus is optionally attached to a PEG molecule linked to chenodeoxvcholate, ursodeoxvcholate, or palmitoyl; or if A1 is 3,3-diphenyl-L-alanine or 3,3-diphenyl-D-alanine, then the N-terminus is attached to palmitoyl;
A2 is L-Thr, L-Ser, L-Val, L-Ala, D-Thr, D-Ser, D-Val, L-tert-leucine, isonipecotic acid, L-α-cyclohexylglycine, bhThr, (2S)-3-hydroxy-2-(methylamino)butanoic acid, D-Ala, L-Cys, D-Cys, L-Pro, D-Pro, or Gly;
A3 is L-His, D-His, 3,3-diphenyl-L-alanine, 3,3-diphenyl-D-alanine, or 2-aminoindane;
A4 is L-Phe, D-Phe, (S)-2-amino-4-phenylbutanoic acid, 3,3-diphenyl-L-alanine, L-biphenylalanine, (1-naphthyl)-L-alanine, (S)-3-Amino-4,4-diphenylbutanoic acid, 4-(aminomethyl)cyclohexane carboxylic acid, (S)-2-amino-3-(perfluorophenyl)propanoic acid, (S)-2-amino-4-phenylbutanoic acid, (S)-2-amino-2-(2,3-dihydro-1H-inden-2-yl)acetic acid, or cyclohexylalanine;
A5 is L-Pro, D-Pro, octahydroindole-2-carboxylic acid, L-β-homoproline, (2S,4S)-4-phenylpyrrolidine-2-carboxylic acid, (2S,5R)-5-phenylpyrrolidine-2-carboxylic acid, or (R)-2-methylindoline;
A6 is L-Ile, D-Ile, L-phenylglycine, L-α-cyclohexylglycine, 4-(aminomethyl)cyclohexane carboxylic acid, (3R)-3-amino-4-methylhexanoic acid, 1-aminocyclohexane-1-carboxylic acid, or (3R)-4-methyl-3-(methylamino)hexanoic acid;
A7 is L-Cys, D-Cys, S-t-Butylthio-L-cysteine, L-homocysteine, L-penicillamine, or D-penicillamine;
A8 is L-Ile, D-Ile, L-α-cyclohexylglycine, 3,3-diphenyl-L-alanine, (3R)-3-amino-4-methylhexanoic acid, 1-aminocyclohexane-1-carboxylic acid, or (3R)-4-methyl-3-(methylamino)hexanoic acid;
A9 is L-Phe, L-Leu, L-Ile, L-Tyr, D-Phe, D-Leu, D-Ile, (S)-2-amino-3-(perfluorophenyl)propanoic acid, N-methyl-phenylalainine, benzylamide, (S)-2-amino-4-phenylbutanoic acid, 3,3-diphenyl-L-alanine, L-biphenylalanine, (1-naphthyl)-L-alanine, (S)-3-amino-4,4-diphenylbutanoic acid, cyclohexylalanine, L-Asp, D-Asp, or cysteamide, wherein L-Phe or D-Phe are optionally linked at the N-terminus to RA, wherein RA is —CONH—CH₂—CH₂—S—, or D-Pro linked to Pro-Lys or Pro-Arg, or L-β-homoproline linked to L-Pro linked to Pro-Lys or Pro-Arg, or D-Pro linked to L-β-homoproline-Lys or L-β-homoproline-Arg; L-Asp or D-Asp are optionally linked at the n-terminus to RB, wherein RB is -(PEG 11)-GYIPEAPRDGQAYVRKDGEWVLLSTFL, or -(PEG 11)-(Gly-Pro-HydroxyPro)₁₀, (S)-2-amino-4-phenylbutanoic acid is linked to RC, wherein RC is D-Pro linked to ProLys or ProArg, or D-Pro linked to L-β-homoproline-Lys or L-β-homoproline-L-Arg;
A10 is L-Cys, L-Ser, L-Ala, D-Cys, D-Ser, or D-Ala;
the carboxy-terminal amino acid is in amide or carboxy-form;
at least one sulfhydryl amino acid is present as one of the amino acids in the sequence; and
A1, A2, A9, A10, or a combination thereof are optionally absent.

A mini-hepcidin of formula A1-A2-A3-A4-A5-A6-A7-A8-A9-A10 or A10-A9-A8-A7-A6-A5-A4-A3-A2-A1 may be a cyclic peptide or a linear peptide.

For example, A1 may be L-Asp; A2, may be L-Th; A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A9 may be L-Phe; A10 may be absent; and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A8 may be L-Ile; A1, A2, A9, and A10 may be absent, and the C-terminus may be amidated. Alternatively, A3 may be L-His; A4 may be L-Phe; A5 may be L-Pro; A6 may be L-Ile; A7 may be L-Cys, D-Cys, S-t-butylthio-L-cysteine, L-homocysteine, L-penicillamine, or D-penicillamine; A1, A2, A8, A9, and A10 may be absent; and the C-terminus may be amidated.

A mini-hepcidin, may comprise the amino acid sequence HFPICI (SEQ ID NO:11), HFPICIF (SEQ ID NO:12), DTHFPICIDTHFPICIF (SEQ ID NO:13), DTHFPIAIFC (SEQ ID NO:14), DTHAPICIF (SEQ ID NO:15), DTHFPICIF (SEQ ID NO:16), or CDTHFPICIF (SEQ ID NO:17). The mini-hepcidin may comprise the sequence set forth in SEQ ID NO:15, for example, wherein the cysteine forms a disulfide bond with S-tertbutyl.

A mini-hepcidin may comprise the amino acid sequence D-T-H-F-P-I-(L-homocysteine)-I-F; D-T-H-F-P-I-(L-penicillamine)-I-F; D-T-H-F-P-I-(D-penicillamine)-I-F; D-(L-tert-leucine)-H-(L-phenylglycine)-(octahydroindole-2-carboxylic acid)-(L-α-cyclohexylglycine)-C-(L-α-cyclohexylglycine)-F; or D-(L-tert-leucine)-H-P-(octahydroindole-2-carboxylic acid)-(L-α-cyclohexylglycine)-C-(L-α-cyclohexylglycine)-F.

A mini-hepcidin may comprise the amino acid sequence FICIPFHTD (SEQ ID NO:18), FICIPFH (SEQ ID NO:19), R2-FICIPFHTD (SEQ ID NO:20), R3-FICIPFHTD (SEQ ID NO:21), FICIPFHTD-R6 (SEQ ID NO:22), R4-FICIPFHTD (SEQ ID NO:23), or R5-FICIPFHTD (SEQ ID NO:24), wherein each amino acid is a D amino acid; R1 is —CONH₂—CH₂—CH₂—S; R2 is chenodeoxycholate-(PEG 11)-; R3 is ursodeoxycholate-(PEG11)-; R4 is palmitoyl-(PEG11)-; R5 is 2(palmitoyl)-diaminopropionic acid-(PEG 11)-; and R6 is (PEG 11)-GYIPEAPRDGQAYVRKDGEWVLLSTFL, wherein each amino acid of R6 is an L amino acid.

A mini-hepcidin may comprise the amino acid sequence D-T-H-((S)-2-amino-4-phenylbutanoic acid)-P-I-C-I-F; D-T-H-(3,3-diphenyl-L-alanine)-P-I-C-I-F; D-T-H-(L-biphenylalanine)-P-I-C-I-F; D-T-H-((1-naphthyl)-L-alanine)-P-I-C-I-F; D-T-H-((S)-3-amino-4,4-diphenylbutanoic acid)-P-I-C-I-F; D-T-H-F-P-I-C-I-((S)-2-amino-4-phenylbutanoic acid); D-T-H-F-P-I-C-I-(3,3-diphenyl-L-alanine); D-T-H-F-P-I-C-I-(L-biphenylalanine); D-T-H-F-P-I-C-I-((1-naphthyl)-L-alanine); D-T-H-F-P-I-C-I-((S)-3-amino-4,4-diphenylbutanoic acid); D-T-H-(3,3-diphenyl-L-alanine)-P-I-C-I-(3,3-diphenyl-L-alanine); D-(3,3-diphenyl-L-alanine)-P-I-C-I-F; D-(3,3-diphenyl-L-alanine)-P-I-C-I-(3,3-diphenyl-L-alanine); D-T-H-(3,3-diphenyl-L-alanine)-P-R-C-R-(3,3-diphenyl-L-alanine); D-T-H-(3,3-diphenyl-L-alanine)-(octahydroindole-2-carboxylic acid)-I-C-I-F; D-T-H-(3,3-diphenyl-L-alanine)-(octahydroindole-2-carboxylic acid)-I-C-I-(3,3-diphenyl-L-alanine); or D-T-H-(3,3-diphenyl-L-alanine)-P-C-C-C-(3,3-diphenyl-L-alanine).

A mini-hepcidin may comprise the amino acid sequence D-T-H-F-P-I-C-I-F-R8; D-T-H-F-P-I-C-I-F-R9; D-T-H-F-P-I-C-I-F-R10; D-T-H-F-P-I-C-I-F-R11; D-T-H-F-P-I-C-I-F-R12; D-T-H-F-P-I-C-I-F-R13; D-T-H-F-P-I-C-I-((S)-2-amino-4-phenylbutanoic acid)-R8; D-T-H-F-P-I-C-I-((S)-2-amino-4-phenylbutanoic acid)-R9; D-T-H-F-P-I-C-I-((S)-2-amino-4-phenylbutanoic acid)-R12; or D-T-H-F-P-I-C-I-((S)-2-amino-4-phenylbutanoic acid)-R13, wherein R8 is D-Pro-L-Pro-L-Lys; R9 is D-Pro-L-Pro-L-Arg; R10 is (L-(3-homoproline)-L-Pro-L-Lys; R11 is (L-β-homoproline)-L-Pro-L-Arg; R12 is D-Pro-(L-β-homoproline)-L-Lys; and R13 is D-Pro-β-homoproline)-L-Arg.

A mini-hepcidin may comprise the amino acid sequence D-T-H-(3,3-diphenyl-L-alanine)-P-(D)R-C-(D)R-(3,3-diphenyl-L-alanine).

A mini-hepcidin may comprise the amino acid sequence C-(isonipecotic acid)-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-(D)P-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide. A mini-hepcidin may comprise the amino acid sequence C-G-(3,3-diphenyl-D-alanine)-(4-(aminomethyl)cyclohexane carboxylic acid)-R-(4-(aminomethyl)cyclohexane carboxylic acid)-(isonipecotic acid)-(3,3-diphenyl-L-alanine)-cysteamide.

A mini-hepcidin may comprise the amino acid sequence (2,2′-azanediyldiacetic acid)-Thr-His-(3,3-diphenyl-L-alanine)-(L-(3-homoproline)-Arg-Cys-Arg-((S)-2-amino-4-phenylbutanoic acid)-(aminohexanoic acid)-(2,2′-azanediyldiacetic acid having a palmitylamine amide on the side chain), which is described in U.S. Pat. No. 9,328,140 (e.g., SEQ ID NO:94 of the '140 patent; hereby incorporated by reference).

In some embodiments, a mini-hepcidin has about 10% to 1000% of the activity of a 25 amino acid long peptide comprising the amino acid sequence set forth in SEQ ID NO:1. For example, a mini-hepcidin may have about 50% to about 200% of the activity of a 25 amino acid long peptide comprising the amino acid sequence set forth in SEQ ID NO:1, such as about 75% to about 150% of the activity, about 80% to about 120% of the activity, about 90% to about 110% of the activity, or about 95% to about 105% of the activity. The term “activity” may refer to the ability of a mini-hepcidin to specifically bind to ferroportin, e.g., thereby inhibiting the transport of intracellular iron into the extracellular space, inhibiting the absorption of dietary iron, and/or reducing serum iron concentration. Activity may refer to the ability of a mini-hepcidin to inhibit the transport of intracellular iron into the extracellular space. Activity may refer to the ability of a mini-hepcidin to inhibit the absorption of dietary iron. Activity may refer to the ability of a mini-hepcidin to reduce serum iron concentration in vivo.

### V. Routes of Administration

The compositions of the invention can be administered in a variety of conventional ways. In some aspects, the compositions of the invention are suitable for parenteral administration. These compositions may be administered, for example, intraperitoneally, intravenously, intrarenally, or intrathecally. In some aspects, the compositions of the invention are injected intravenously. One of skill in the art would appreciate that a method of administering a therapeutically effective substance formulation or composition of the invention would depend on factors such as the age, weight, and physical condition of the patient being treated, and the disease or condition being treated. The skilled worker would, thus, be able to select a method of administration optimal for a patient on a case-by-case basis.

The composition may be administered topically, enterally, or parenterally. The composition may be administered subcutaneously, intravenously, intramuscularly, intranasally, by inhalation, orally, sublingually, by buccal administration, topically, transdermally, or transmucosally. The composition may be administered by injection. In preferred embodiments, the composition is administered by subcutaneous injection, orally, intranasally, by inhalation, or intravenously. In certain preferred embodiments, the composition is administered by subcutaneous injection.

Throughout this specification, the word “comprise” or variations such as “comprises” or “comprising” will be understood to imply the inclusion of a stated integer (or components) or group of integers (or components), but not the exclusion of any other integer (or components) or group of integers (or components). The singular forms “a,” “an,” and “the” include the plurals unless the context clearly dictates otherwise. The term “including” is used to mean “including but not limited to.” “Including” and “including but not limited to” are used interchangeably. The terms “patient” and “individual” are used interchangeably and refer to either a human or a non-human animal. These terms include mammals such as humans, primates, livestock animals (e.g., bovines, porcines), companion animals (e.g., canines, felines) and rodents (e.g., mice, rabbits and rats).

“About” and “approximately” shall generally mean an acceptable degree of error for the quantity measured given the nature or precision of the measurements. Typically, exemplary degrees of error are within 20%, preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

As used herein, the term “administering” means providing a pharmaceutical agent or composition to a subject, and includes, but is not limited to, administering by a medical professional and self-administering. Such an agent, for example, may be hepcidin or a hepcidin analogue.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, a therapeutic that “prevents” a condition (e.g., iron overload) refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

In certain embodiments, agents of the invention may be used alone or conjointly administered with another type of therapeutic agent. As used herein, the phrase “conjoint administration” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control.

While specific aspects of the patient matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations. Experiments relating to the therapeutic effect of hepcidin and related compounds are published in PCT application WO 2017/120419.

What is claimed is:

1. A method for treating a condition in a subject, comprising administering a composition comprising hepcidin or mini-hepcidin to the subject.

2. The method of claim 1, wherein administering a composition to the subject comprises administering about 10 μg to about 1 gram of hepcidin or mini-hepcidin.

3. The method of claim 2, wherein administering a composition to the subject comprises administering about 100 μg to about 100 mg of hepcidin or mini-hepcidin.

4. The method of claim 3, wherein administering a composition to the subject comprises administering about 200 μg to about 50 mg of hepcidin or mini-hepcidin.

5. The method of claim 4, wherein administering a composition to the subject comprises administering about 500 μg to about 10 mg of hepcidin or mini-hepcidin.

6. The method of claim 5, wherein administering a composition comprising hepcidin or mini-hepcidin to the subject comprises administering about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin.

7. The method of any one of the preceding claims, wherein administering a composition to the subject comprises administering a bolus of the composition.

8. The method of any one of the preceding claims, wherein administering the composition comprises administering the composition at least once per month.

9. The method of claim 8, wherein administering the composition comprises administering the composition at least once per week.

10. The method of claim 9, wherein administering the composition comprises administering the composition 1, 2, 3, 4, 5, 6, or 7 times per week.

11. The method of claim 10, wherein administering the composition comprises administering the composition 1, 2, or 3 times per week.

12. The method of any one of claims 8 to 11, wherein about 10 μg to about 1 gram of hepcidin or mini-hepcidin is administered each time the composition is administered.

13. The method of claim 12, wherein about 100 μg to about 100 mg of hepcidin or mini-hepcidin is administered each time the composition is administered.

14. The method of claim 13, wherein about 200 μg to about 50 mg of hepcidin or mini-hepcidin is administered each time the composition is administered.

15. The method of claim 14, wherein about 500 μg to about 10 mg of hepcidin or mini-hepcidin is administered each time the composition is administered.

16. The method of claim 15, wherein about 500 μg, about 600 μg, about 667 μg, about 700 μg, about 750 μg, about 800 μg, about 850 μg, about 900 μg, about 950 μg, about 1000 μg, about 1200 μg, about 1250 μg, about 1300 μg, about 1333 μg, about 1350 μg, about 1400 μg, about 1500 μg, about 1667 μg, about 1750 μg, about 1800 μg, about 2000 μg, about 2200 μg, about 2250 μg, about 2300 μg, about 2333 μg, about 2350 μg, about 2400 μg, about 2500 μg, about 2667 μg, about 2750 μg, about 2800 μg, about 3 mg, about 3.3 mg, about 3.5 mg, about 3.7 mg, about 4 mg, about 4.5 mg, about 5 mg, about 6 mg, about 7 mg, about 8 mg, about 9 mg, or about 10 mg of hepcidin or mini-hepcidin is administered each time the composition is administered.

17. The method of any one of claims 1 to 16, wherein the composition is administered subcutaneously, intravenously, intramuscularly, intranasally, by inhalation, orally, sublingually, by buccal administration, topically, transdermally, or transmucosally.

18. The method of any one of claims 1 to 17, wherein the composition is administered by injection.

19. The method of claim 17, wherein the composition is administered intravenously.

20. The method of any one of claims 1 to 19, wherein the condition is α-thalassemia, thalassemia intermedia, β-thalassemia, hemochromatosis, sickle cell disease, refractory anemia, or hemolytic anemia.

21. The method of claim 20, wherein the condition is anemia and the anemia is a hemoglobinopathy, sideroblastic anemia, anemia associated with myelodysplastic syndrome (MDS), or a congenital anemia.

22. The method of claim 20, wherein the condition is anemia and the anemia is Diamond-Blackfan anemia.

23. The method of any one of claims 1 to 19, wherein the condition is hepatocarcinoma, cardiomyopathy, or diabetes.

24. The method of any one of claims 1 to 19, wherein the condition is a viral, bacterial, fungal, or protist infection.

25. The method of claim 24, wherein the condition is abdominal sepsis or a systemic infection.

26. The method of claim 24, wherein the condition is a bacterial infection, and the bacteria is Escherichia coli, Neisseria cinerea, Neisseria gonorrhoeae, Staphylococcus epidermidis, Staphylococcus aureus, or Streptococcus agalactiae.

27. The method of claim 24, wherein the condition is a bacterial infection, and the bacteria are Mycobacterium (such as M. africanum, M. avium, M. tuberculosis, M. bovis , M. canetti, M. kansasii, M. leprae, M. lepromatosis, or M. microti).

28. The method of claim 24, wherein the condition is a bacterial infection, and the bacteria are gram-negative bacteria.

29. The method of claim 28, wherein the gram-negative bacteria are Acetic acid bacteria, Acinetobacter Baumann, Bdellovibrio, Borrelia, Bortadella, Burkhoderia, Chlamydia, Cyanobacteria, Campylobacter, Citrobacter, Enterobacter, Neisseria meningitidis, Fusobacterium, green sulfur bacteria, green non-sulfur bacteria, Haemophilus Influenzae, Helicobacter, Hemophilus, Klebsiella, Legionella, Leptospiria, Moraxella, Nitrobacter, Pseudomonas, Proteus, Rickettsia, Serratia, Salmonella, Shigella, spirochaetes, Stenotrophomonas, Thiobacter, Treponema, Vibrio, and/or Yersinia.

30. The method of claim 24, wherein the condition is a fungal infection, and the fungus is Candida albicans.

31. The method of claim 24, wherein the condition is a fungal infection, and the fungal infection is Mucormycosis.

32. The method of claim 24, wherein the condition is a protist infection, and the protist is Trypanosoma cruzi, Plasmodium (such as P. falciparum, P. vivax, P. ovale, or P. malariae), Trypanosoma brucei (such as T. brucei gambiense or T. brucei rhodesiense), or Leishmania.

33. The method of claim 24, wherein the condition is a protist infection, and the protist is Naegleria.

34. The method of any one of claims 1 to 19, 24, and 32, wherein the condition is Chagas disease, malaria, African sleeping sickness, or leishmaniasis.

35. The method of claim 24, wherein the condition is a viral infection, and the virus is hepatitis B, hepatitis C, or dengue virus.

36. The method of claim 24, wherein the condition is a bacterial infection and the bacterial infection is tuberculosis.

37. The method of any one of the preceding claims, wherein the subject is a mammal.

38. The method of claim 37, wherein the subject is a rodent, lagomorph, feline, canine, porcine, ovine, bovine, equine, or primate.

39. The method of claim 38, wherein the subject is a human.

40. The method of claim 39, wherein the subject has a serum hepcidin concentration of less than about 100 ng/mL prior to administering the composition.

41. The method of claim 40, wherein the subject has a serum hepcidin concentration of less than 50 ng/mL prior to administering the composition.

42. The method of any one of claims 39 to 41, wherein the subject has a serum ferritin concentration greater than 100 ng/mL prior to administering the composition.

43. The method of claim 42, wherein the subject has a serum ferritin concentration greater than 1000 ng/mL prior to administering the composition.

44. The method of any one of claims 39 to 43, wherein the subject has a total body iron content of about 40 to about 50 mg/kg prior to administering the composition.

45. The method of any one of claims 39 to 43, wherein the subject has a total body iron content greater than 50 mg/kg prior to administering the composition.

46. The method of claim 45, wherein the subject has a total body iron content greater than 60 mg/kg prior to administering the composition.

47. The method of any one of claims 39 to 46, wherein the serum iron concentration of the subject is at least about 100 μg/dL prior to administering the composition.

48. The method of claim 47, wherein the serum iron concentration of the subject is at least about 200 μg/dL prior to administering the composition.

49. The method of any one of claims 39 to 48, wherein the transferrin saturation of the subject is greater than about 20% prior to administering the composition to the subject.

50. The method of claim 49, wherein the transferrin saturation of the subject is greater than about 50% prior to administering the composition to the subject.

51. The method of any one of claims 1 to 50, wherein the composition comprises hepcidin and the hepcidin comprises the amino acid sequence set forth in SEQ ID NO:1, SEQ ID NO:2, SEQ ID NO:3, SEQ ID NO:4, or SEQ ID NO:5.

52. The method of any one of claims 1 to 50, wherein the composition comprises hepcidin and the hepcidin comprises an amino acid sequence having at least 90% sequence homology with the amino acid sequence set forth in SEQ ID NO:1, SEQ ID NO:2, SEQ ID NO:3, SEQ ID NO:4, or SEQ ID NO:5.

53. The method of claim 52, wherein the hepcidin comprises each of the 8 cysteines in SEQ ID NO:1, SEQ ID NO:2, SEQ ID NO:3, SEQ ID NO:4, or SEQ ID NO:5.

54. The method of claim 51 or 53, wherein the 8 cysteines in SEQ ID NO:1, SEQ ID NO:2, SEQ ID NO:3, SEQ ID NO:4, or SEQ ID NO:5 form 4 disulfide bonds in the hepcidin.

55. The method of any one of claims 51 to 54, wherein the hepcidin comprises the amino acid sequence set forth in SEQ ID NO:1.

56. The method of any one of claims 1 to 50, wherein the composition comprises hepcidin and the hepcidin comprises the sequence set forth in SEQ ID NO:6, SEQ ID NO:7, SEQ ID NO:8, SEQ ID NO:9, or SEQ ID NO:10.

57. The method of claim 56, wherein the 8 cysteines of SEQ ID NO:6, SEQ ID NO:7, SEQ ID NO:8, SEQ ID NO:9, or SEQ ID NO:10 form 4 disulfide bonds in the hepcidin.

58. The method of any one of claims 1 to 50, wherein the composition comprises a mini-hepcidin.

59. The method of any one of claims 1 to 19, wherein the condition is malaria.

60. The method of claim 59, wherein the malaria is a drug-resistant strain of malaria.

61. The method of claim 59, wherein the composition comprising mini-hepcidin or hepcidin is conjointly administered with an antimalarial drug.

62. The method of claim 61, wherein the antimalarial drug is selected from tetracycline, proguanil, chlorproguanil, pyronaridine, lumefantrinel, mefloquine, dapsone, atovaquone, artesunate, and artemisinin.

63. The method of claim 59, wherein the subject has a G6PD deficiency.

64. A method of overcoming, preventing, or inhibiting the development of drug resistance in a subject with malaria, comprising administering to the subject an antimalarial drug and a composition comprising hepcidin or mini-hepcidin.

65. The method of claim 64, wherein the antimalarial drug is selected from tetracycline, proguanil, chlorproguanil, pyronaridine, lumefantrinel, mefloquine, dapsone, atovaquone, artesunate, and artemisinin.

66. A method of treating or preventing a parasitic worm infection in a subject, comprising conjointly administering to the subject an antiparasitic drug and a composition comprising hepcidin or mini-hepcidin.

67. The method of claim 66, wherein the parasitic worm infection is caused by a fluke.

68. The method of claim 66 or 67, wherein the parasitic worm infection is fascioliasis.

69. The method of claim 66 or 67, wherein the parasitic worm infection is paragonimiasis.

70. The method of any one of claims 66 to 69, wherein the antiparasitic drug is an anthelmintic.

71. The method of any one of claims 66 to 70, wherein the antiparasitic drug is triclabendazole, nitazoxanide, metronidazole, niclofolan, chloroquine, artesunate, armamentarium, praziquantel, bithionol, emetine, dehydroemetine, or derivatives thereof.

72. The method of claim 71, wherein the antiparasitic drug is triclabendazole.

73. The method of claim 66 or 67, wherein the subject has a drug resistant parasitic worm infection.

74. The method of claim 73, wherein the drug resistant parasitic worm is triclabendazole-resistant fascioliasis.

75. A method of treating or preventing fascioliasis in a subject, comprising conjointly administering to the subject triclabendazole and a composition comprising hepcidin or mini-hepcidin.

76. A method of overcoming, preventing, or inhibiting the development of drug resistance in a subject with fascioliasis, comprising administering to the subject an antiparasitic drug and a composition comprising hepcidin or mini-hepcidin.

77. The method of claim 76, wherein the antiparasitic drug is triclabendazole.

78. A pharmaceutical composition comprising an antimalarial drug and an agent selected from hepcidin and mini-hepcidin.

79. The pharmaceutical composition of claim 78, wherein the antimalarial drug is selected from tetracycline, proguanil, chlorproguanil, pyronaridine, lumefantrinel, mefloquine, dapsone, atovaquone, artesunate, and artemisinin.

80. The pharmaceutical composition of claim 78 or 79, wherein the antimalarial drug is artesunate.

81. The pharmaceutical composition of any one of claims 78 to 80, wherein the pharmaceutical composition is suitable for administration subcutaneously, intravenously, intramuscularly, intranasally, by inhalation, orally, sublingually, by buccal administration, topically, transdermally, or transmucosally.

82. The pharmaceutical composition of any one of claims 78 to 81, wherein the pharmaceutical composition is suitable for administration by injection.

83. The pharmaceutical composition of any one of claims 78 to 82, wherein the pharmaceutical composition is suitable for administration intravenously.

84. The pharmaceutical composition of any one of claims 78 to 81, wherein the pharmaceutical composition is suitable for administration orally.

85. A method of treating or preventing malaria in a subject, comprising administering to the subject the pharmaceutical composition of any one of claims 78 to 84.

86. The method of claim 85, wherein the malaria is a drug-resistant strain of malaria.

87. A method of overcoming, preventing, or inhibiting the development of drug resistance in a subject with malaria, comprising administering to the subject the pharmaceutical composition of any one of claims 78 to 84.

88. The method of any one of claims 85 to 87, wherein the pharmaceutical composition is administered subcutaneously, intravenously, intramuscularly, intranasally, by inhalation, orally, sublingually, by buccal administration, topically, transdermally, or transmucosally.

89. The method of any one of claims 85 to 88, wherein the pharmaceutical composition is administered by injection.

90. The method of any one of claims 85 to 89, wherein the pharmaceutical composition is administered intravenously.

91. The method of any one of claims 85 to 88, wherein the pharmaceutical composition is administered orally.

92. The method of any one of claims 1 to 19, wherein the condition is pneumonia.

93. The method of claim 92, wherein the pneumonia is bacterial pneumonia, viral pneumonia, fugal pneumonia, or parasitic pneumonia.

94. The method of claim 92 or 93, wherein the subject has a drug-resistant pneumonia.

95. The method of 94, wherein the subject has an antibiotic-resistant pneumonia.

96. The method of claim 92-95, wherein the pneumonia is bacterial pneumonia.

97. The method of claim 92-96, wherein the composition comprising hepcidin or mini-hepcidin further comprises or is conjointly administered with one or more antibiotics.

98. The method of claim 97, wherein the one or more antibiotics are selected from macrolides, ketolides, fluoroketolides, tetracyclines, fluoroquinolones, cephalosporins, penicillins, atovaquone, Trimethoprim-sulfamethoxazole, and vancomycin.

99. The method of claim 92-94, wherein the pneumonia is viral pneumonia.

100. The method of claim 99, wherein the composition comprising hepcidin or mini-hepcidin further comprises or is conjointly administered with an antiviral drug.

101. The method of claim 100, wherein the antiviral drug is selected from amantadine, rimantadine, zanamivir, acyclovir, ribavarin, and oseltamivir.

102. The method of claim 92-94, wherein the pneumonia is fungal pneumonia.

103. The method of claim 102, wherein the composition comprising hepcidin or mini-hepcidin further comprises or is conjointly administered with an antifungal drug.

104. The method of claim 103, wherein the antifungal drug is selected from amphotericin B, voriconazole, posaconazole, fluconazole, itraconazole, flucytosine, ketoconazole, triazoles, echinocandins, isavuconazole, atovaquone, and caspofungin.

105. The method of claim 92-94, wherein the pneumonia is parasitic pneumonia.

106. The method of claim 105, wherein the composition comprising hepcidin or mini-hepcidin further comprises or is conjointly administered with an antiparasitic drug.

107. The method of claim 106, wherein the antiparasitic drug is an anthelmintic.
